(19) United States
(12) Patent Application Publication
(10) Pub. No.: US 2022/0055989 A1
(43) Pub. Date: Feb. 24, 2022

(54) CONDENSED PYRROLES AS NOVEL BROMODOMAIN INHIBITORS

(71) Applicant: ALBERT LUDWING UNIVERSITÄT FREIBURG, Freiburg (DE)

(72) Inventors: Stefan Gunther, Freiburg (DE); Daniel Wohlwend, Karlsruhe (DE); Martin Hugle, Freiburg (DE); Xavier Lucas, Dundee (GB); Pierre-Michel Regenass, Charrat (CH); Mehrosh Pervaiz, Freiburg (DE); Roben Warstat, Freiburg (DE); Bernhard Breit, Gundelfingen (DE); Oliver Einsle, Freiburg (DE)

(21) Appl. No.: 17/417,071

(22) PCT Filed: Dec. 19, 2019
(86) PCT No.: PCT/EP19/86482

§ 371 Date: Jun. 21, 2021

(30) Foreign Application Priority Data
Dec. 21, 2018 (EP) 18215794.1

(51) Int. Cl. C07D 209/52; A61K 45/06; A61K 31/403; A61K 31/5377; A61K 31/496; A61K 31/55; C07D 403/12

(52) U.S. Cl. CPC C07D 209/52; A61K 45/06; A61K 31/403; C07D 403/12; A61K 31/496; A61K 31/55; A61K 31/5377

(57) ABSTRACT

Compounds of formula (1) or (2) and their use in the treatment of diseases or conditions for which a bromodomain inhibitor is indicated.

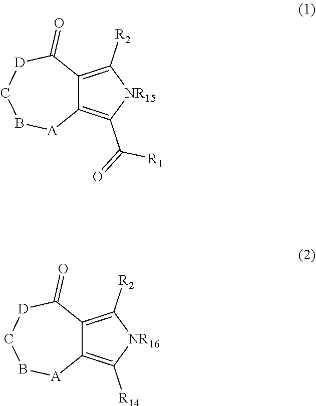

The present invention relates to novel compounds which may be used as bromodomain inhibitors.

Bromodomains are epigenetic reader modules regulating gene transcription by recognizing acetyl-lysine modified histone tails.

The human genome encodes up to 61 different bromodomains (BRDs), present in transcriptional co-regulators and chromatin modifying enzymes including histone acetyl-transferases (HATs) and the Bromodomain extra-terminal domain (BET) family. They are epigenetic mark ‘readers’ that specifically recognize ε-N-acetylated lysine residues (K_{ac}).

Drug molecules that target epigenetic mechanisms of gene regulation are attractive as they offer the perspective of modifying processes responsible for dysfunctional malignant states rather than just treating the results thereof.

Bromodomains are small distinct domains within proteins that bind to acetylated lysine residues commonly but not exclusively in the context of histones.

BRDs fold into an evolutionary conserved four anti-parallel helix motif, linked by diverse loop regions of variable length (ZA and BC loops), which define the K_{ac} binding site (Filippakopoulos et al. (2012), Cell 149(1), 214-231). In most BRDs, this site features an asparagine residue mainly responsible for substrate recognition (Owen D J, et al. (2000), The structural basis for the recognition of acetylated histone H4 by the bromodomain of histone acetyltransferase gcn5p, The EMBO journal 19(22):6141-6149; Umehara T, et al. (2010) Structural basis for acetylated histone H4 recognition by the human BRD2 bromodomain, The Journal of biological chemistry 285(10):7610-7618).

The biological function of BRDs and their potential as therapeutic targets have been thoroughly described in literature (Muller S, Filippakopoulos P, & Knapp S (2011), Bromodomains as therapeutic targets, Expert reviews in molecular medicine 13:e29; Prinjha R K, Witherington J, & Lee K (2012), Place your BETs: the therapeutic potential of bromodomains, Trends in pharmacological sciences 33(3):146-153).

Research on the structural classification of bromodomains has revealed that the BET family of bromodomains has an overall highly targetable recognition site (Vidler L R, Brown N, Knapp S., Hoelder S (2012), Druggability analysis and structural classification of bromodomain acetyl-lysine binding sites, Journal of medicinal chemistry 55(17):7346-7359).

Members of the BET family, namely BRD2, BRD3, BRD4, and BRDT, modulate gene expression by recruiting transcriptional regulators to specific genomic locations. BRD4 and BRD2 have crucial roles in cell cycle control of mammalian cells. Along with BRD3, they are functionally linked to pathways important for cellular viability and cancer signalling and are co-regulators in obesity and inflammation. Specifically, BRD4 has been characterized as a key determinant in acute myeloid leukaemia, multiple myeloma, Burkitt's lymphoma, NUT midline carcinoma, colon cancer, and inflammatory disease. Because of its continued association with K_{ac} in mitotic chromosomes, BRD4 has been postulated to be important for the maintenance of epigenetic memory.

Small molecules that inhibit bromodomains have potential as anti-inflammatory, antiviral, and anticancer agents. Anticancer activity is mainly due to down-regulation of the key oncogene c-MYC Recently, cytotoxicity in LAC cells has been related to suppression of the oncogenic transcription factor FOSL1 and its targets.

US 2015/111890 relates to isoindolone derivatives of the formula

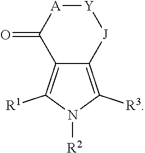

wherein A, Y and J each are a substituted or unsubstituted methylene group, R¹ is hydrogen or a C₁-C₃ alkyl group, R² is hydrogen or a C₁ to C₃ alkyl group and R³ is an aryl or heteroaryl group. The compounds are said to show activity as bromodomain inhibitors.

US 2016/0200681 discloses on page 7 as intermediates in a reaction scheme compounds of formula

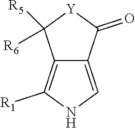

wherein Y is a C₁-C₃ alkylene group or —NH—, R₁ is i.a. a C₁-C₄ alkyl group and R₅ and R₆ are hydrogen or C₁-C₄ alkyl groups. The compounds are used for the treatment of peptic ulcer, gastritis or reflux esophagitis.

WO 2014/170350 relates to compounds of the formulae

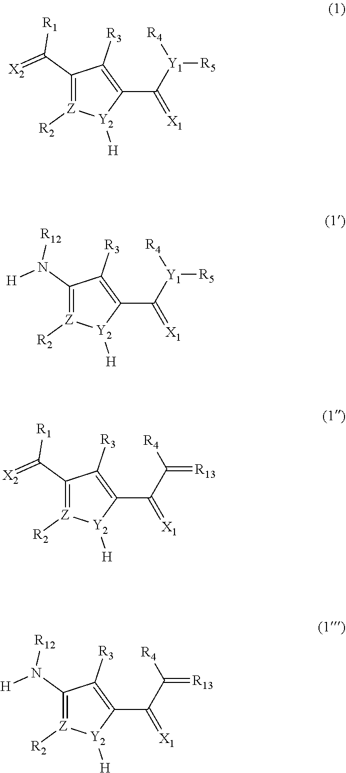

or a pharmaceutically acceptable salt thereof for use in the treatment of diseases or conditions for which a bromodomain inhibitor is indicated

There is still a need for novel bromodomain inhibitors which may be used in the treatment of a variety of diseases.

Accordingly, it has been an object of the present invention to provide novel bromodomain inhibitors.

This object has been achieved with the compounds as defined in claim 1.

Preferred embodiments of the present invention are set forth in the dependent claims and in the detailed specification hereinafter.

Furthermore, another embodiment of the present invention relates to the use of the novel compounds of formula (I) as bromodomain inhibitors.

The novel compounds in accordance with the present invention are represented by formula (1) or (2)

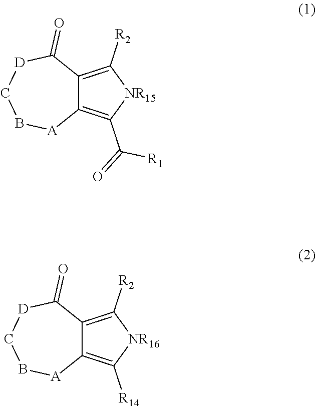

wherein

A, B, C and D, which may be the same or different, independent of one another represent —C(R₇R₈)—, —C(R₉R₁₀)—C(R₁₁R₁₂)—, —N(R₁₃)—, —O— or —S— with the proviso that the total number of ring atoms of the ring comprising ring members A, B, C and D is 7 to 9,

R₁ is selected from the group consisting of OH, OR₃, NH₂, NHR₄ and NR₅R₆

R₂ is a substituted or unsubstituted alkyl group with 1 to 8 carbon atoms or a substituted or unsubstituted alkoxy group with 1 to 8 carbon atoms in which one or more of the hydrogen atoms may be optionally replaced by halogen,

R₃ and R₄, independent of each other, represent an alkyl group with 1 to 6 carbon atoms or a substituted or unsubstituted alkylaryl, aryl or heteroaryl group,

R₅ and R₆, independent of each other, represent an alkyl group with 1 to 6 carbon atoms or a substituted or unsubstituted aryl or heteroaryl group or form together with the nitrogen atom to which they are attached, a six- or seven membered, substituted or unsubstituted heterocyclic ring, R₇ to R₁₃, independent of each other, represent hydrogen, hydroxyl, substituted or unsubstituted alkyl groups with 1 to 4 carbon atoms, substituted or unsubstituted alkoxy groups with 1 to 4 carbon atoms and substituted or unsubstituted hydroxyalkyl groups with 1 to 4 carbon atoms or wherein two of R₇ to R₁₃ located at adjacent atoms form together a chemical bond or a ring,

R₁₄ represents a substituted or unsubstituted aryl or heteroaryl group, and R₁₅ and R₁₆, independent of each other, are hydrogen or a C₁-C₄-alkyl group, preferably R₁₅ and R₁₆ represent hydrogen.

The term substituted or unsubstituted divalent alkylene group with 2 to 4 carbon atoms, when used herein, represents a group —(CR′R″)ₙ— wherein n is an integer from 2 to 4 and R′ and R″, independent of each other represent hydrogen, a C₁ to C₄ alkyl group or a C₁-C₄ alkoxy group.

The term C₁-Cₙ alkyl group, when used herein, represents a linear or branched hydrocarbon group with 1 to n carbon atoms. Examples are methyl, ethyl, i-propyl, n-propyl, i-butyl, n-butyl and t-butyl, to name only a few representatives.

One or more hydrogen atoms in the alkyl groups can be replaced by substituents consisting of halogen, alkyl, alkoxy, amino, cyano, alkenyl, alkynyl, arylalkyl, aryl, and heteroaryl groups.

The term C₁ to C₈ alkoxy group, when used herein, represents a derivative of an alkyl group comprising at least one O—C bond. Representative examples are OCH₃, OC₂H₅, OC₃H₇ and OC₄H₉. One or more hydrogen atoms attached to the carbon atoms in the alkoxy groups can be replaced by substituents consisting of halogen, alkyl, alkoxy, amino, cyano, alkenyl, alkynyl, arylalkyl, aryl, and heteroaryl groups.

The term C₁-C₈ hydroxyalkyl group, when used herein, represents a linear or branched C₁-C₈-alkyl group in which one or more of the hydrogen atoms are replaced by a hydroxy group. One or more of the remaining hydrogen atoms attached to the carbon atoms in the hydroxyalkyl groups can be replaced by substituents consisting of halogen, alkyl, alkoxy, amino, cyano, alkenyl, alkynyl, arylalkyl, aryl, and heteroaryl groups.

In accordance with a preferred embodiment, at least one, preferably at least two, more preferably at least three of groups A, B, C and D represent a group —(CR₇R₈)— or a methylene group (—CH₂—). Particularly preferred A, B, C and D are all methylene groups.

In accordance with another preferred embodiment R₂ is an alkyl group with 1 to 4 carbon atoms, preferably a methyl group, an ethyl group, a propyl group or a butyl group. In some cases a methyl group has been found to be advantageous.

R₁ in the compounds of formula (1) preferably is a group OR₃, NHR₄ or a substituted aryl group. The aryl group is preferably a phenyl group, which may be unsubstituted or may carry one or more substituents. Preferred substituents of the aryl, respectively phenyl groups are alkoxy groups, groups —S(═O)₂—R with R being preferably an amino group, a heterocyclic ring with 4 to 7 carbon atoms or a carboxamido group.

If R₁ is OR₃ or NHR₄, R₃ and R₄ preferably represent an alkyl group with 1 to 4 carbon atoms.

A preferred group of substituents R₄ in compounds of formula (1) is represented by formula (3)

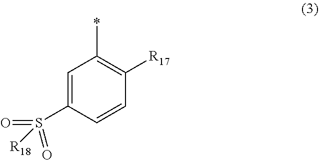

wherein * represents the site of attachment to the nitrogen atom,

R₁₇ is hydroxyl or an alkoxy group with 1 to 4 carbon atoms and R₁₈ is —NR₁₉R₂₀ wherein R₁₉ and R₂₀, independent of each other, represent an alkyl group with 1 to 4 carbon atoms, a substituted or unsubstituted aryl ring or together with the nitrogen atom to which they are attached form a substituted or unsubstituted heterocyclic ring with 4 to 7 ring atoms.

In accordance with still another preferred embodiment, R₁₄ in the compounds of formula (2) is a substituted or unsubstituted phenyl, oxadiazole or indole group. Preferred substituents at these rings are amino, hydroxyl, halogen, carboxamide and alkyl groups.

Further preferred compounds of formula (1) in accordance with the present invention are represented by formulae (4) to (7)

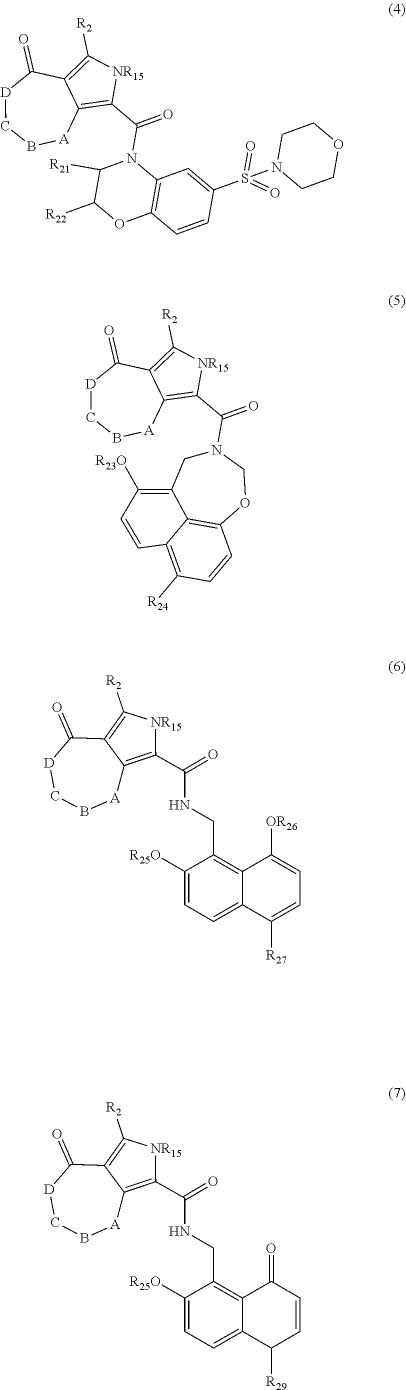

wherein A, B, C, D, R₂ and R₁₅ are as defined in claim 1,

R₂₁ and R₂₂, independent of each other, are hydrogen, C₁-C₄ alkyl or form together a chemical bond,

R₂₃, R₂₅ and R₂₈, independent of each other, are hydrogen or C₁ to C₄ alkyl,

R₂₄, R₂₇ and R₂₉, independent of each other, are C₁-C₄ alkyl or C₂-C₄-carboxyl, and

R₂₆ is hydrogen or C₁ to C₄ alkyl.

The preferred embodiments for the substituents in formula (4) to (7) are as defined for the preferred embodiments described hereinbefore.

Preferred examples of compounds of formula (2) in accordance with the present invention are represented by formula (2′)

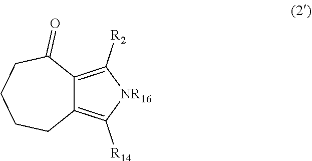

wherein R₂, R₄ and R₁₆ are as defined in claim 1.

In the compounds of formula (2) and (2′), R₂ is preferably an alkyl group with 1 to 4 carbon atoms. R₁₄ is preferably a substituted or unsubstituted phenyl or a substituted oxadiazole group. R₁₆ is preferably hydrogen. Preferred substituents of the phenyl or oxadiazole group are OH, NH₂, halogen, carboxamide or alkyl. The phenyl or oxadiazole groups in the preferred compounds of formula (2′) may carry one or more than one substituent, with a combination of halogen and amino groups being particularly preferred (such as e.g. in compounds (21), (26) and (27) below).

The following compounds (8) to (27) represent particularly preferred embodiments of the compounds of the present invention, wherein formulae (8) to (18) are representative of compounds of formula (1) and formulae (19) to (27) are representative of compounds of formula (2):

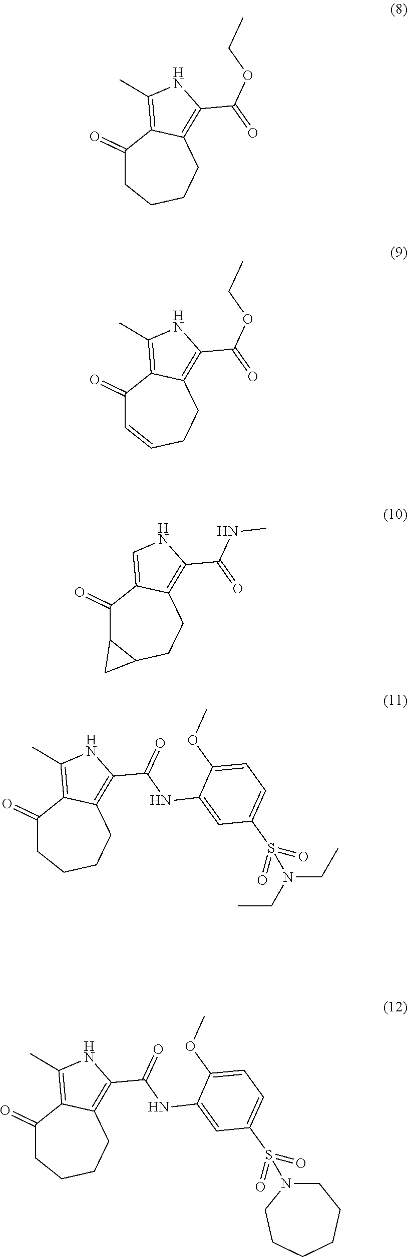 [SMILES: CCOC(=O)c1[nH]c(C)c2c1CCCCC2=O ; CCOC(=O)c1[nH]c(C)c2c1CCC=CC2=O ; CNC(=O)c1[nH]cc2c1CCC1CC1C2=O ; CCN(CC)S(=O)(=O)c1ccc(OC)c(NC(=O)c2[nH]c(C)c3c2CCCCC3=O)c1 ; COc1ccc(S(=O)(=O)N2CCCCCC2)cc1NC(=O)c1[nH]c(C)c2c1CCCCC2=O]

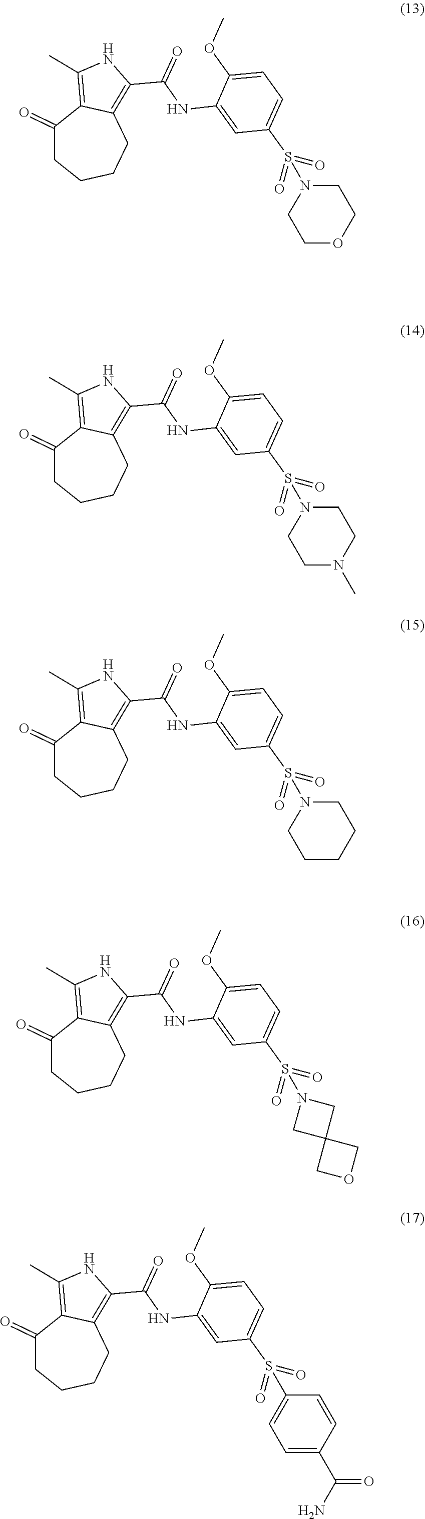 [SMILES: COc1ccc(S(=O)(=O)N2CCOCC2)cc1NC(=O)c1[nH]c(C)c2c1CCCCC2=O ; COc1ccc(S(=O)(=O)N2CCN(C)CC2)cc1NC(=O)c1[nH]c(C)c2c1CCCCC2=O ; COc1ccc(S(=O)(=O)N2CCCCC2)cc1NC(=O)c1[nH]c(C)c2c1CCCCC2=O ; COc1ccc(S(=O)(=O)N2CC3(COC3)C2)cc1NC(=O)c1[nH]c(C)c2c1CCCCC2=O ; COc1ccc(S(=O)(=O)c2ccc(C(N)=O)cc2)cc1NC(=O)c1[nH]c(C)c2c1CCCCC2=O]

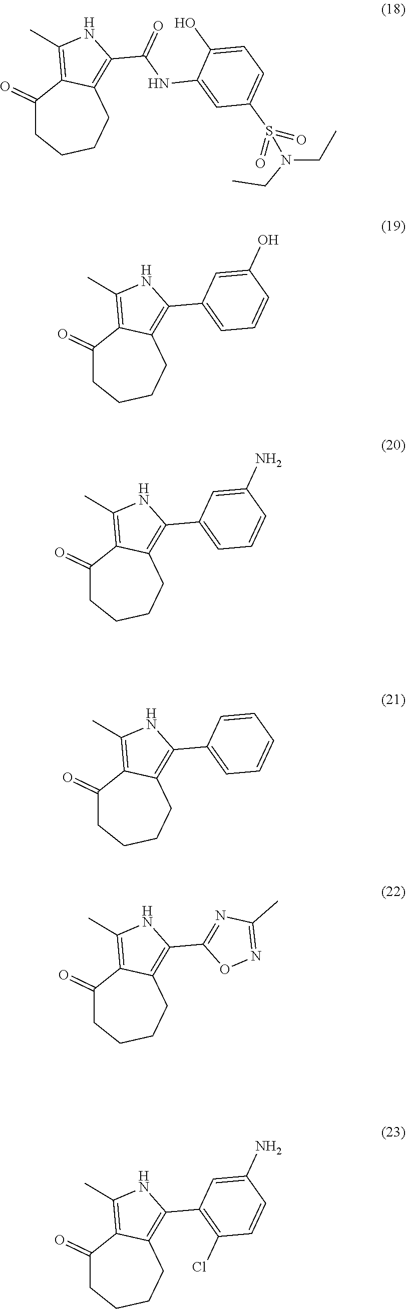 [SMILES: CCN(CC)S(=O)(=O)c1ccc(O)c(NC(=O)c2[nH]c(C)c3c2CCCCC3=O)c1 ; Cc1[nH]c(-c2cccc(O)c2)c2c1C(=O)CCCC2 ; Cc1[nH]c(-c2cccc(N)c2)c2c1C(=O)CCCC2 ; Cc1[nH]c(-c2ccccc2)c2c1C(=O)CCCC2 ; Cc1noc(-c2[nH]c(C)c3c2CCCCC3=O)n1 ; Cc1[nH]c(-c2cc(N)ccc2Cl)c2c1C(=O)CCCC2]

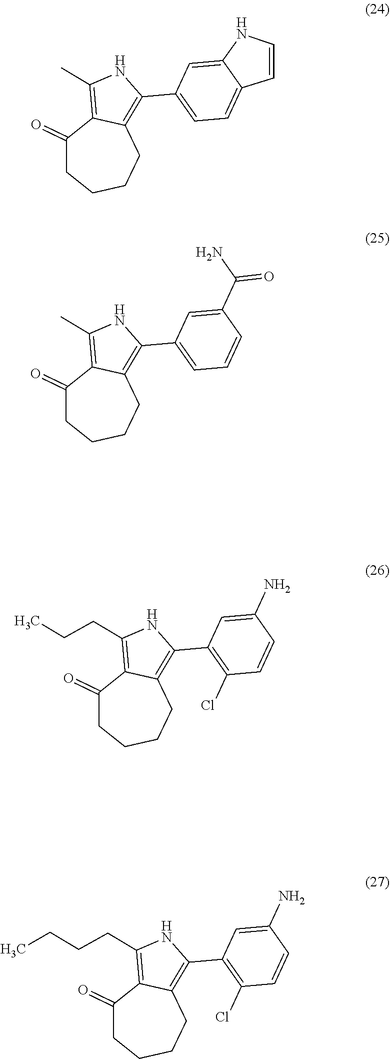 [SMILES: Cc1[nH]c(-c2ccc3cc[nH]c3c2)c2c1C(=O)CCCC2 ; Cc1[nH]c(-c2cccc(C(N)=O)c2)c2c1C(=O)CCCC2 ; CCCc1[nH]c(-c2cc(N)ccc2Cl)c2c1C(=O)CCCC2 ; CCCCc1[nH]c(-c2cc(N)ccc2Cl)c2c1C(=O)CCCC2]

Another group of preferred compounds of formula (1) are represented by formula (1′)

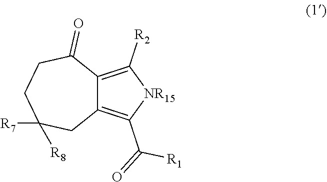

wherein R₁, R₂, R₇, R₈ and R₁₅ are as defined in claim 1.

In accordance with another preferred embodiment, R₂ is a substituted or unsubstituted alkyl group with 1 to 4 carbon atoms.

In accordance with still another preferred embodiment, R₁ is a group NHR₆ wherein R₆ is as defined in claim 1.

In accordance with another preferred embodiment, R₁₅ is hydrogen.

A particularly preferred compound of formula (1) in accordance with the present invention is represented by formula (1″)

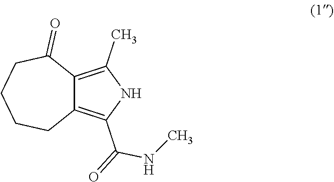 [SMILES: CNC(=O)c1[nH]c(C)c2c1CCCCC2=O]

In accordance with the present invention the compounds of formula (1) or (2) may be used in the form of their pharmaceutically acceptable salts. The skilled person is aware of pharmaceutically acceptable salts of compounds of formula (1) or (2) and will select the appropriate salt based on his professional experience and knowledge. Suitable pharmaceutically acceptable salts can include acid or base addition salts. For a review on suitable salts see Berge et al. (1977) J. Pharm. Sci. 66:1-19. Typically, pharmaceutical acceptable salts may be readily prepared by using a desired acid or base as appropriate. The resultant salt may precipitate from solution and be collected by filtration or may be recovered by evaporation of the solvent.

A pharmaceutically acceptable base addition salt can be formed by reaction of a compound of formula (1) or (2) with a suitable inorganic or organic base, optionally in a suitable solvent, to give the base addition salt which is usually isolated, for example by crystallization and filtration. Pharmaceutically acceptable base salts include ammonium salts, alkali metal salts, alkaline earth metal salts and salts with organic basis, in particular salt with primary, secondary and tertiary amines, e.g. isopropyl amine, diethylamine, ethanolamine, trimethylamine and dicyclohexylamine.

The pharmaceutically acceptable acid addition salt can be formed by reaction of a compound of formula (1) or (2) with a suitable inorganic or organic acid, in a suitable solvent such as an organic solvent, to give the product which is usually isolated, for example, by crystallization and filtration. Pharmaceutically acceptable acid addition salts of a compound of formula (I) can comprise or can be, for example, a nitrate, sulphate, hydrobromide, hydrochloride, phosphate, maleate, acetate, propionate, fumarate, citrate, tartrate, salicylate, aspartate, succinate, benzoate, p-toluenesulphonate, methanesulphonate, naphthalenesulphonate, ethanesulphonate or hexanoate salt.

In accordance with the present invention the pharmaceutically acceptable salts include all possible stoichiometric and non-stoichiometric forms of the salts of the compounds of formula (1) or (2).

The compounds of formula (1) or (2) form complexes with solvents in which they are reacted or from which they are precipitated or crystallized, which complexes are generally referred to as solvates. In accordance with the present invention all possible stoichiometric and non-stoichiometric forms of the solvates of the compounds of formula (1) or (2) are included.

The present invention also encompasses all prodrugs of the compounds of formula (1) or (2) and pharmaceutically acceptable salts thereof, which upon administration to the recipient are capable of providing (directly or indirectly) a compound of formula (1) or (2) or a pharmaceutically acceptable salt thereof or an active metabolite or acceptable salt thereof. In this regard, reference is made to Burger's Medicinal Chemistry and drug discovery, 5th Ed., Vol. 1, Principles and Practice, to which reference is made here for further details.

The compounds of formula (1) or (2) may be in crystalline or amorphous form. Furthermore, some of the crystalline forms may exist in different morphological conformations, which are included within the scope of the present invention. Different morphological forms (polymorphic forms) of the compounds of formula (1) or (2) can be characterized and different analytical techniques are known to the person skilled in the art so that no further details are necessary here.

The compounds of formula (1) or (2) may also exist in one of several tautomeric forms and in accordance with the present invention individual tautomers or mixtures thereof are encompassed.

The compounds of formula (1) or (2) or their pharmaceutically acceptable salts in accordance with another embodiment of the present invention are used in the treatment of diseases or conditions for which a bromodomain inhibitor is indicated.

Bromodomain inhibitors are believed to be useful in the treatment of a variety of diseases or conditions related to systemic or tissue inflammation, inflammatory responses to infection or hypoxia, cellular activation and proliferation, lipid metabolism, fibrosis and in the prevention and treatment of viral infections.

Bromodomain inhibitors may thus be useful in the treatment of a great variety of chronic autoimmune and inflammatory conditions such as rheumatoid arthritis, osteoarthritis, acute gout, psoriasis, systemic lupus erythematosus, multiple sclerosis, inflammatory bowel disease, asthma, chronic obstructive airways disease, pneumonitis, myocarditis, pericarditis, myositys, eczema, dermatitis, alopecia, vitiligo, bullous skin diseases, nephritis, vasculitis, atherosclerosis, Alzheimer's disease, depression, retinitis, uveitis, scleitis, hepatitis, pancreatitis, primary biliary cirrhosis, sclerosis, cholangitis, Addison's disease, hypophysitis, thyroiditis, type I diabetes and acute rejection of transplanted organs.

The compounds in accordance with formula (1) or (2) may also be useful in the prevention or treatment of diseases or conditions which involve inflammatory responses to infections with bacteria, viruses, fungi, parasites or the like, such as sepsis, sepsis syndrome, septic shock, endotoxemia, systemic inflammatory response syndrome (SIRS), multi-organ dysfunction syndrome, toxic shock syndrome, acute lung injury, adult respiratory distress syndrome, acute renal failure, fulminant hepatitis, burns, acute pancreatitis, post-surgical syndromes, sarcoidosis, Herxheimer reactions, encephalitis, myelitis, meningitis, malaria, and SIRS associated with viral infections such as influenza, herpes zoster, herpes simplex, and coronavirus.

The compounds of formula (1) or (2) may also be used in the treatment of disorders of the lipid metabolism involving the regulation of APO-A1, such as hypercholesterolemia, atherosclerosis, and Alzheimer's disease.

Furthermore, the compounds of formula (1) or (2) may be used in the prevention or treatment of conditions associated with ischemia-reperfusion injury such as myocardial infarction, cerebrovascular ischemia (stroke), acute coronary syndromes, renal reperfusion injury, organ transplantation, coronary artery bypass grafting, cardio-pulmonary bypass procedures and pulmonary, renal, hepatic, gastro-intestinal or peripheral limb embolism.

Bromodomain inhibitors may further be useful in the treatment of fibrotic conditions such as idiopathic pulmonary fibrosis, renal fibrosis, post-operative stricture, keloid formation, scleroderma and cardiac fibrosis.

A further indication for bromodomain inhibitors is the prevention and treatment of viral infections such as herpes virus, human papilloma virus, adenovirus, poxvirus and other DNA viruses.

The compounds of formula (1) or (2) are useful in the treatment of cancer, including but not limited to types of cancer included in the NCI60 panel, such as bladder cancer, brain cancer, breast cancer, cervical carcinoma, colorectal cancer, oesophageal cancer, gastric cancer, head and neck cancer, leukaemia, lymphoma, NSCLC cancer (non-small cell lung carcinoma), ovarian cancer, pancreatic cancer, sarcoma, SCLC cancer (small cell lung cancer), melanoma, renal cancer, prostate cancer and hepatocellular carcinoma. including haematological (such as leukaemia), epithelial including lung, breast and colon carcinomas, midline carcinomas, mesenchyme, hepatic, renal and neurological tumours.

Preferred uses of the compounds of formula (1) or (2) are the treatment of chronic autoimmune or inflammatory conditions, cancer or viral diseases.

The compounds of formula (1) or (2) can also be preferably used for the inhibition of proliferation of leukaemia cells or for the treatment of a disease or condition which is obesity or a kidney malfunction.

Furthermore, the compounds of formula (1) or (2) could also be effective as male contraceptives.

The compounds of formula (1) or (2) can also be preferably used for the treatment of diseases caused by parasites such as malaria or leishmaniasis.

Compounds of formula (1) or (2) can also be used in the treatment of diseases or conditions related to bromodomains CREBBP and EP300. By way of example, acute myeloid leukaemia, acute lymphoblastic leukaemia, non-Hodgkin lymphoma, prostate cancer and spinal and bulbar muscular atrophy may be mentioned in this regard.

In the course of the studies leading to the present invention it has been recognized that the compounds of formula (1) or (2) in particular are effective in inhibiting the bromodomains of the BET family, including BRD2-BD1, BRD2-BD2, BRD3-BD1, BRD3-BD2, BRD4-BD1, BRD4-BD2 BRDT-BD1, BRDT-BD2 as well as the bromodomains, BRD7, BRD9, BRPF1, BAZ2A, BAZ2B, BRD1, BRD8-BD1, CECR2, FALZ, PBRM1-BD2, BPRM1-BDS, TAF1-BD2, TAF1L-BD2, TRIM24, TRIM33, CREBBP and EP300, with a particularly good effect in the inhibition of the mentioned bromodomains of the BET family. In some cases, particularly good results were obtained in the inhibition of bromodomains BRD7 and BRD9.

In accordance with the present invention, the compounds of formula (1) or (2) as well as their pharmaceutically acceptable salts may be administered as such or may be presented as active ingredients in a pharmaceutical composition.

Accordingly, a further embodiment of the present invention relates to a pharmaceutical composition comprising at least one compound of formula (1) or (2) or a pharmaceutically acceptable salt thereof and one or more pharmaceutically acceptable carriers, diluents or excipients.

The carriers, diluents or excipients used in pharmaceutical compositions must be acceptable in the sense of being compatible with the other ingredients of the composition and not detrimental to the recipient.

Such pharmaceutical compositions may be obtained by mixing a compound of formula (1) or (2) or a pharmaceutically acceptable salt thereof, with one or more pharmaceutically acceptable carriers, diluents or excipients.

Since the compounds and pharmaceutically acceptable salts thereof are intended for use in pharmaceutical compositions it is apparent that they are preferably provided in substantially pure form, i.e. with a degree of impurities as low as possible. Preferably the purity is at least 60%, more preferably at least 75% and most preferably at least 98% (in each case percent by weight).

Pharmaceutical compositions may be presented in unit dose forms containing a predetermined amount of active ingredient per unit dose. Preferred unit dosage compositions are those containing a daily dose or sub-dose, or an appropriate fraction thereof, of an active ingredient. Such unit doses may therefore be administered more than once a day. Preferred unit dosage compositions are those containing a daily dose or sub-dose as recited above.

The pharmaceutical compositions may be adapted for administration by any appropriate route, for example by the oral, rectal, inhalation, intranasal, topical, vaginal or parenteral route.

The skilled person will select the appropriate administration route based on the conditions of the specific application. The manufacture of the appropriate administration forms is known to the skilled person so that no further details are necessary here.

When desired or necessary, suitable binders, glidants, lubricants, sweetening agents, flavours, disintegrating agents and colouring agents can be included in the pharmaceutical composition. The skilled person will select the appropriate additives based on his professional experience and in adaptation to the specific application.

The pharmaceutical compositions in accordance with the present invention may comprise more than one active ingredient, e.g. more than one compound of formula (1) or (2) or a pharmaceutically acceptable salt thereof or a combination of a compound of formula (1) or (2) with other active ingredients or their pharmaceutically acceptable salts.

In particular combinations of bromodomain inhibitors of formula (1) or (2) and histone deacetylase inhibitors (HDAC inhibitors) provide a very interesting property spectrum and combination of efficiencies.

Various HDAC inhibitors useful to be combined with the compounds of formula (1) or (2) have been investigated and described in the literature and are known to the skilled person.

Vorinostat and Romidepsin have been approved by the US FDA for cutaneous T-cell lymphoma.

Panobinostat, valproic acid and belinostat are in phase III clinical trials for various types of cancer.

Mocetinostat, Abexinostat, Entinostat, Resminostat, Givinostat, Qusinostat and SB 939 are in phase II clinical trials.

CUDC-101, AR-42, ACY-1215, Kevetrin, Trichostatin A are in early stage development as HDAC inhibitors.

All the aforementioned HDAC inhibitors can principally be combined with the compounds of formula (1) or (2) in accordance with the present invention to obtain pharmaceutical compositions.

The compounds of formula (1) or (2) for use in accordance with the present invention present a novel class of interesting active molecules in the epigenetic field and in particular may be considered as a representative of a new class of bromodomain inhibitors of the BET family.

The skilled person is aware of suitable methods for the synthesis of compounds of formula (1) or (2) so that there is no further description necessary here.

The compounds of formula (1) or (2) or a pharmaceutically acceptable salt thereof may be used in the manufacture of a medicament for the treatment of diseases or conditions for which a bromodomain inhibitor is indicated. In other embodiments, there is provided the use of a compound of formula (1) or (2) or a pharmaceutically acceptable salt thereof in the manufacture of a medicament for the treatment of a chronic autoimmune and/or inflammatory condition or in the treatment of cancer.

In a further embodiment there is provided a method for treatment of a disease or condition, for which a bromodomain inhibitor is indicated, in a subject in need thereof which comprises administering a therapeutically effective amount of a compound of formula (1) or (2) or a pharmaceutically acceptable salt thereof.

In one embodiment the subject in need of treatment is a mammal.

The term effective amount means an amount of a drug or pharmaceutical agent that will elicit the biological or medical response of a tissue, system, animal or human that is being sought, for instance by a researcher or a clinician. Furthermore, the term therapeutically effective amount means any amount which, as compared to a corresponding subject which has not received such amount, results in improved treatment, healing, prevention or amelioration of a disease, disorder, or side effect, or a decrease in the rate of advancement of a disease or disorder. The term also includes within its scope amounts effective to enhance normal physiological function.

The following examples represent embodiments of the present invention.

The skilled person will easily recognize that he can modify the structure of the compounds in the working examples in an appropriate manner.

### EXAMPLES

General Procedures

Reagents and solvents were obtained from commercial sources and used without any further purification.

Column chromatography was accomplished using MACHEREY-NAGEL silica gel 60® (230-400 mesh). Thin layer chromatography was performed on aluminum plates pre-coated with silica gel (MERCK, 60F254), which were visualized by UV fluorescence (λₘₐₓ=254 nm) and/or by staining with ninhydrin in ethanol (EtOH).

¹H and ¹³C NMR Spectroscopy

NMR spectra were acquired on a BRUKER Avance 400 spectrometer (400

MHz and 100.6 MHz for ¹H and ¹³C respectively) or a Bruker 500 DRX NMR spectrometer with TBI probe head (499.6 MHz and 125.6 MHz for ¹H and ¹³C respectively) at a temperature of 303 K unless specified. Chemical shifts are reported in parts per million (ppm) relative to residual solvent. Data for ¹H NMR are described as following: chemical shift (δ in ppm), multiplicity (s, singlet; d, doublet; t, triplet; q, quartet; m, multiplet; br, broad signal), coupling constant (Hz), integration. Data for ¹³C NMR are described in terms of chemical shift (δ in ppm).

High Resolution Mass Spectrometry (HR-MS) data were obtained on a THERMO SCIENTIFIC Advantage and a THERMO SCIENTIFIC instrument (Atmospheric Pressure Chemical Ionization (APCI)/methanol (MeOH): spray voltage 4-5 kV, ion transfer tube: 250-300° C., vaporizer: 300-400° C.).

### Example 1 Synthesis of 3-methyl-4-oxo-2,4,5,6,7,8-hexahydrocyclohepta[c]pyrrole-1-carboxylic acid

General Synthesis Scheme

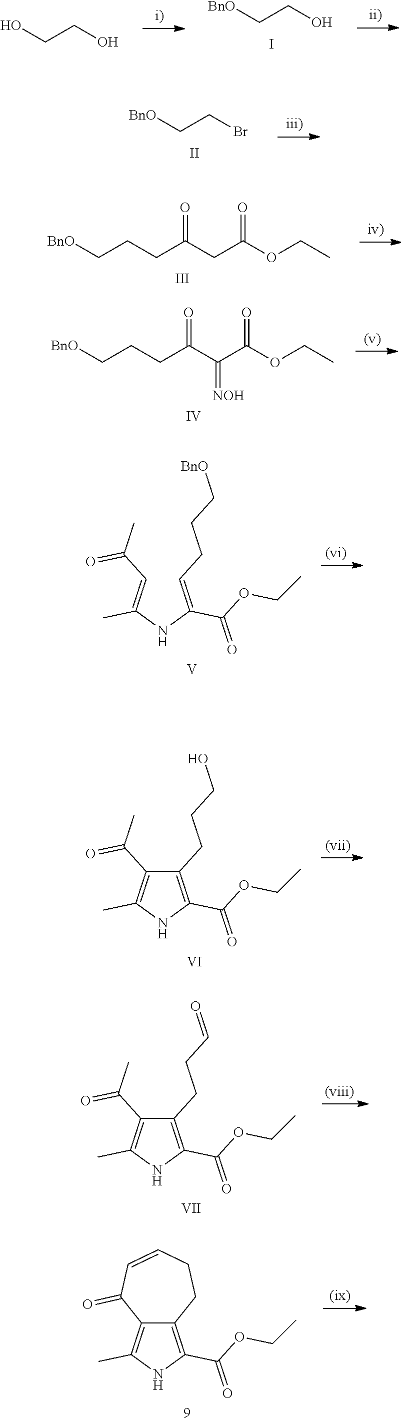

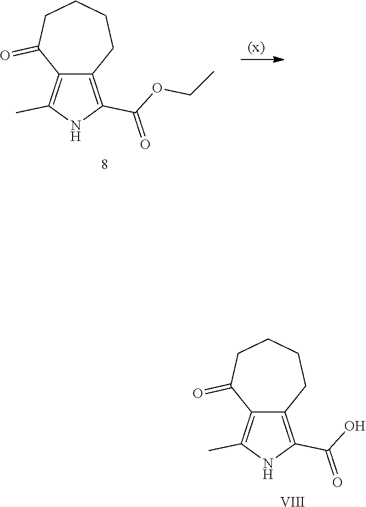

Reaction conditions: i) Benzyl bromide (BnBr), sodium hydride (NaH), THF, room temperature (room temperature, hereinafter referred to as rt), 93%; ii) N-Bromosuccinimide (NBS), triphenylphosphine (PPh₃), dichloromethane (CH₂Cl₂), −78° C. to rt, 89%; iii) Ethyl acetoacetate, NaH, n-butyllithium (BuLi), −20° C. to rt, 76%; iv) sodium nitrite (NaNO₂), acetic acid (AcOH)/H₂O, 0° C. to rt, 96%; v) Pentane-2,4-dione, Zn, AcOH, sodium acetate (AcONa), 90° C., 63%; vi) H₂, palladium on charcoal (Pd/C), ethyl acetate (EtOAc)/(MeOH), rt, 67%; vii) (COCl)₂, triethylamine multiplicity (s, singlet; d, doublet; t, triplet; q, quartet; m, multiplet; br, broad signal), coupling constant (Hz), integration. Data for ¹³C NMR are described in terms of chemical shift (δ in ppm).

High Resolution Mass Spectrometry (HR-MS) data were obtained on a THERMO SCIENTIFIC Advantage and a THERMO SCIENTIFIC instrument (Atmospheric Pressure Chemical Ionization (APCI)/methanol (MeOH): spray voltage 4-5 kV, ion transfer tube: 250-300° C., vaporizer: 300-400° C.).

### Example 1 Synthesis of 3-methyl-4-oxo-2,4,5,6,7,8-hexahydrocyclohepta[c]pyrrole-1-carboxylic acid

General Synthesis Scheme

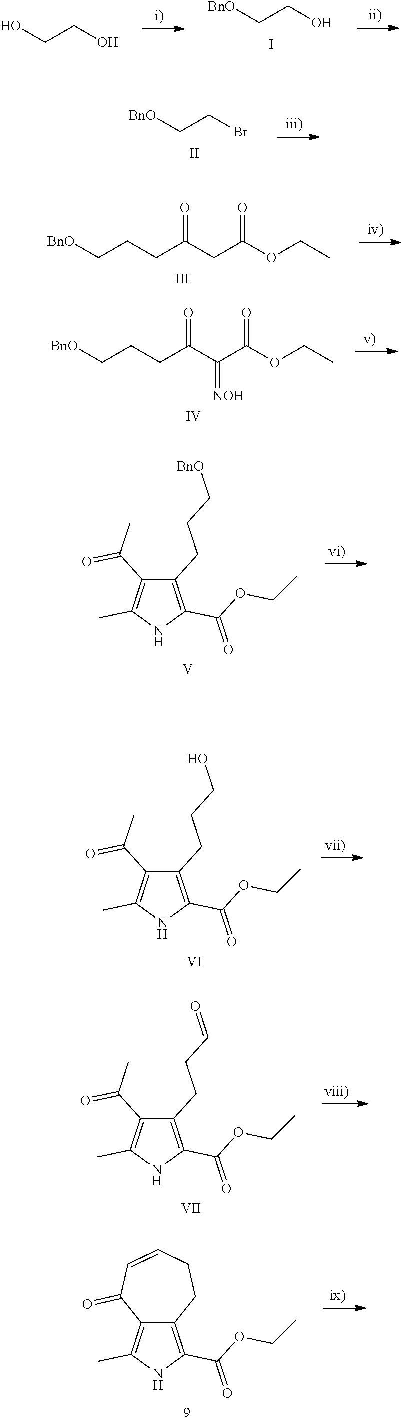

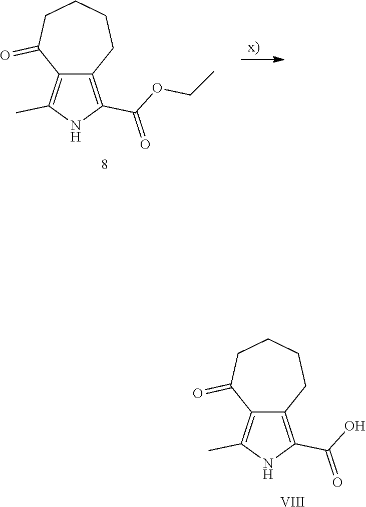

Reaction conditions: i) Benzyl bromide (BnBr), sodium hydride (NaH), THF, room temperature (room temperature, hereinafter referred to as rt), 93%; ii) N-Bromosuccinimide (NBS), triphenylphosphine (PPh₃), dichloromethane (CH₂Cl₂), −78° C. to rt, 89%; iii) Ethyl acetoacetate, NaH, n-butyllithium (BuLi), −20° C. to rt, 76%; iv) sodium nitrite (NaNO₂), acetic acid (AcOH)/H₂O, 0° C. to rt, 96%; v) Pentane-2,4-dione, Zn, AcOH, sodium acetate (AcONa), 90° C., 63%; vi) H₂, palladium on charcoal (Pd/C), ethyl acetate (EtOAc)/(MeOH), rt, 67%; vii) (COCl)₂, triethylamine (NEt₃), DMSO, NEt₃, CH₂Cl₂, −78° C. to rt, 100%; viii) p-toluene sulfonic acid (PTSA).H₂O, toluene (PhMe), 110° C., 56%; ix) H₂, Pd/C, EtOAc/MeOH, rt, 98%; x) NaOH, EtOH/H₂O, reflux, then HCl, 0° C., 71%

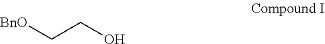 [SMILES: OCCOCc1ccccc1]

2-(benzyloxy)ethan-1-ol (Compound I). To a suspension of NaH (10.2 g, 0.25 mmol, 1.05 equiv) in dry THF (152 mL) ethylene glycol (250 g, 2.42 mol, 10 equiv) was added dropwise at 0 C. The reaction mixture was first stirred at rt for 30 min and afterwards benzylbromide (48 mL, 0.24 mol, 1.0 equiv) was added at 0 C. The mixture was stirred for 12 h at rt and afterwards quenched with saturated ammonium chloride (NH₄Cl) solution. The aqueous phase was extracted three times with diethyl ether (Et₂O). The combined organic layers were washed with H₂O, brine, dried over sodium sulfate (Na₂SO₄) and the solvent was evaporated under reduced pressure. The residue was purified by column chromatography (20-100% EtOAc in petroleum ether (hereinafter referred to as PE) to afford the desired compound as a light yellow oil (56.7 g, 373 mmol, 93%).

¹H NMR (400 MHz, CDCl₃) δ 0.39-7.27 (m, 5H), 4.56 (s, 2H), 3.79-3.69 (m, 2H), 3.61-3.54 (m, 2H), 2.61-2.50 (m, 1H); ¹³C NMR (101 MHz, CDCl₃) δ 138.0, 128.4, 127.8, 127.7, 73.2, 71.5, 61.8; R_{f}: 0.39 (40% AcOEt in PE).

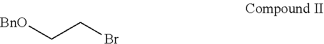 [SMILES: BrCCOCc1ccccc1]

((2-bromoethoxy)methyl)benzene (Compound II). To a solution of NBS (17.7 g, 99.3 mol, 1 equiv.) in CH₂Cl₂ (200 mL) was added dropwise a solution of PPh₃ (26.1 g, 99.3 mol, 1 equiv.) in CH₂Cl₂ (140 mL) at −78° C. The solution was stirred for 1 h, then a solution of 2-(benzyloxy)ethan-1-ol (14.4 g, 94.6 mmol, 1 equiv.) in CH₂Cl₂ (100 mL) was added dropwise. The solution was allowed to return to rt and after one hour, the reaction was quenched using MeOH (10 mL) and PhMe (150 mL). The solution was evaporated, water was added and the aqeuous phase was extracted three times with EtOAc. The combined organics were washed with brine, dried over Na₂SO₄ and evaporated. The residue was purified on silica gel column eluting with 10% EtOAc in cyclohexane to afford the desired product (18.1 g, 84.1 mol, 89%) as a light yellow oil.

¹H NMR (400 MHz, CDCl₃) δ 7.41-7.28 (m, 5H), 4.60 (s, 2H), 3.80 (t, J=6.2 Hz, 2H), 3.50 (t, J=6.2 Hz, 2H); ¹³C NMR (101 MHz, CDCl₃) δ 137.8, 128.5, 127.9, 127.8, 73.2, 70.0, 30.5; R_{f}: 0.85 (20% AcOEt in CyH)

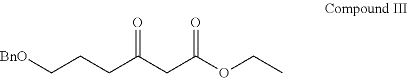 [SMILES: CCOC(=O)CC(=O)CCCOCc1ccccc1]

Ethyl 6-(benzyloxy)-3-oxohexanoate (Compound III). Ethyl acetoacetate (32 mL, 252 mmol, 1.0 equiv) was added dropwise to a suspension of NaH (5.55 g, 278 mmol, 1.1 equiv, 60% w/w) in dry THF (500 mL) at −20 C. The reaction mixture was stirred for 1 h, then BuLi in hexane (2.5 M, 112 mL, 278 mmol, 1.1 equiv) was added dropwise. After 1 h, ((2-bromoethoxy)methyl)benzene (65.1 g, 302 mmol, 1.0 equiv) was added and the reaction mixture was allowed to return to rt and stirred overnight. A saturated NH₄Cl solution was added and the organic phase was evaporated. The aqueous phase was extracted three times with EtOAc. The combined organic layers were washed with brine, dried over Na₂SO₄ and evaporated. The residue was purified by column chromatography eluting with 10-100% EtOAc in PE to afford the desired compound as an orange liquid (50.5 g, 191 mmol, 76%). ¹H NMR (300 MHz, CDCl₃) δ 7.41-7.28 (m, 5H), 4.50 (s, 2H), 4.20 (q, J=7.1 Hz, 2H), 3.51 (t, J=6.1 Hz, 2H), 3.45 (s, 2H), 2.68 (t, J=7.1 Hz, 2H), 1.99-1.89 (m, 2H), 1.29 (t, J=7.1 Hz, 3H); ¹³C NMR (101 MHz, CDCl₃) δ 202.6, 167.3, 138.5, 128.5, 127.7, 127.7, 73.0, 69.1, 61.4, 49.5, 39.8, 23.8, 14.2; HRMS (ESI): calcd. for C₁₅H₂₀O₄Na [M+Na]⁺: 287.1254, found: 287.1254; R_{f}: 0.30 (20% AcOEt in cyclohexane (CyH)).

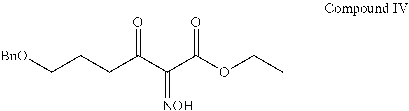

Ethyl (Z)-6-(benzyloxy)-2-(hydroxyimino)-3-oxohexanoate (Compound IV). To a solution of ethyl-6-(benzyloxy)-3-oxohexanoate (63.0 g, 238 mmol, 1.0 equiv) in AcOH (240 mL) at 0 C, was added dropwise a solution of NaNO₂ (32.9 g, 477 mmol, 2.0 equiv) in H₂O (125 mL). The mixture was then allowed to return to rt, and stirred overnight. Water was added and the aqueous phase was extracted four times with ethyl acetate (EA). The combined organic layers were washed three times with saturated Na₂CO₃ solution, brine and dried over Na₂SO₄ to afford the desired compound as a yellow oil (67.0 g, 229 mmol, 96%). ¹H NMR (400 MHz, CDCl₃) δ 10.51 (s, 1H), 7.42-7.26 (m, 5H), 4.53 (s, 2H), 4.35 (q, J=7.1 Hz, 2H), 3.55 (td, J=6.1, 1.7 Hz, 2H), 2.87 (dt, J=35.4, 7.2 Hz, 2H), 2.09-1.78 (m, 2H), 1.33 (t, J=7.1 Hz, 3H); ¹³C NMR (101 MHz, CDCl₃) δ 195.6, 161.9, 150.6, 137.5, 128.5, 127.9, 127.9, 73.0, 69.2, 62.2, 34.4, 23.6, 14.0; HRMS (ESI): calcd. for C₁₅H₁₉NO₅Na [M+Na]⁺: 316.1155, found: 316.1155; R_{f}: 0.63 (33% AcOEt in CyH).

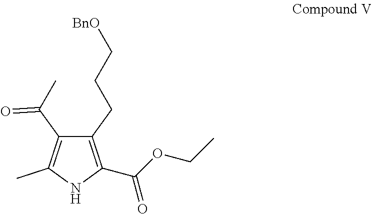 [SMILES: CCOC(=O)c1[nH]c(C)c(C(C)=O)c1CCCOCc1ccccc1]

Ethyl 4-acetyl-3-(3-(benzyloxy)propyl)-5-methyl-1-H-pyrrole-2-carboxylate (Compound V). To a solution of ethyl (Z)-6-(benzyloxy)-2-(hydroxyimino)-3-oxohexanoate (28.1 g, 95.9 mmol, 0.8 equiv.) and pentane-2,4-dione (12.0 g, 120 mmol, 1 equiv.) in AcOH (274 mL) was added AcONa (39.3 g, 479 mmol, 4 equiv.). The solution was heated to 90° C. and Zn (10.5 g, 161 mmol, 3 equiv.) was added portionwise. The mixture was then heated to 90° C. for 1 h, filtered and evaporated. The residue was dissolved in water and the aqueous phase was extracted three times with EtOAc. The combined organics were washed with brine, dried over Na₂SO₄ and evaporated. The residue was purified on silica gel column eluting with 10-50% EtOAc in cyclohexane to give the desired product (20.9 g, 60.7 mmol, 63%) as a colorless solid. ¹H NMR (400 MHz, CDCl₃) δ 9.48 (s, 1H), 7.40-7.27 (m, 5H), 4.51 (s, 2H), 4.31 (q, J=7.2 Hz, 2H), 3.55 (t, J=6.6 Hz, 2H), 3.21-3.07 (m, 2H), 2.52 (s, 3H), 2.46 (s, 3H), 1.93-1.78 (m, 2H), 1.33 (t, J=7.1 Hz, 3H); ¹³C NMR (101 MHz, CDCl₃) δ 195.3, 161.7, 138.9, 138.0, 134.2, 128.3, 127.6, 127.4, 123.1, 118.0, 72.8, 70.5, 60.5, 31.4, 31.1, 22.7, 15.3, 14.4; HRMS (ESI): calcd. for C₂₀H₂₅NO₄Na [M+Na]⁺: 366.1676, found: 366.1676; R_{f}: 0.49 (40% EtOAc in cyclohexane (CyH)).

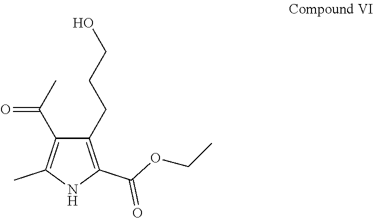 [SMILES: CCOC(=O)c1[nH]c(C)c(C(C)=O)c1CCCO]

Ethyl 4-acetyl-3-(3-hydroxypropyl)-5-methyl-1H-pyrrole-2-carboxylate (Compound VI). To a solution of ethyl 4-acetyl-3-(3-(benzyloxy)propyl)-5-methyl-1H-pyrrole-2-carboxylate (2.40 g, 6.99 mmol, 1 equiv.) in EtOAc and MeOH (28 mL, 1:1) under argon, was added Pd/C (580 mg, 5% w/w). The solution was put under H₂ (1 atm) and stirred overnight at rt. The mixture was filtered over celite, evaporated and purified on silica gel column eluting with 50-100% EtOAc in CyH to afford the desired product (1.19 g, 4.70 mmol, 67%) as a light yellow solid. ¹H NMR (400 MHz, CDCl₃) δ 9.92 (s, 1H), 4.31 (q, J=7.2 Hz, 2H), 3.53 (dd, J=6.1, 5.3 Hz, 2H), 3.36 (br s, 1H), 3.15 (t, J=6.9 Hz, 2H), 2.54 (s, 3H), 2.44 (s, 3H), 1.89-1.72 (m, 2H), 1.34 (t, J=7.1 Hz, 3H); ¹³C NMR (101 MHz, CDCl₃) δ 195.8, 161.6, 138.1, 134.5, 123.2, 118.4, 61.1, 60.6, 33.5, 31.1, 21.3, 15.5, 14.4; HRMS (ESI): calcd. for C₁₃H₁₉NO₄Na [M+Na]⁺: 276.1206, found: 276.1206; R_{f}: 0.42 (80% EtOAc in CyH).

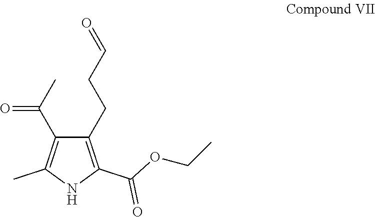 [SMILES: CCOC(=O)c1[nH]c(C)c(C(C)=O)c1CCC=O]

Ethyl 4-acetyl-5-methyl-3-(3-oxopropyl)-1H-pyrrole-2-carboxylate (Compound VII). To a solution of DMSO (374 μl, 3.16 mmol, 2 equiv.) in dry CH₂Cl₂ (10 mL) was added (0001)₂ (203 μl, 2.37 mmol, 1.5 equiv.) at −78° C. dropwise. The mixture was stirred at −78° C. for 15 min, then a solution of ethyl 4-acetyl-3-(3-hydroxypropyl)-5-methyl-1H-pyrrole-2-carboxylate (400 mg, 1.58 mmol, 1 equiv.) in dry CH₂Cl₂ (5.8 mL) was added dropwise. After 30 min, NEt₃ (1.10 mL, 7.90 mmol, 5 equiv.) was added. The mixture was stirred 30 min at −78° C., then, was allowed to return to rt. The solution was evaporated and water was added. The aqueous phase was extracted three times with EtOAc and evaporated to afford after fast purification on silica gel column (20-100% EtOAc in cyclohexane), the desired product (397 mg, 1.58 mmol, 100%) as a colorless oil. ¹H NMR (400 MHz, CDCl₃) δ 9.81 (t, J=2.0 Hz, 1H), 9.07 (s, 1H), 4.33 (q, J=7.1 Hz, 2H), 3.40 (dd, J=8.0, 7.1 Hz, 2H), 2.74-2.65 (m, 2H), 2.44 (s, 3H), 1.35 (t, J=7.1 Hz, 3H); ¹³C NMR (101 MHz, CDCl₃) δ 202.7, 194.7, 161.1, 137.2, 132.8, 123.1, 118.4, 60.7, 44.8, 31.2, 19.1, 15.7, 14.5; HRMS (ESI): calcd. for C₁₃H₁₆NO₄ [M−H]⁻: 250.1085, found: 250.1085; R_{f}: 0.50 (50% AcOEt in CyH).

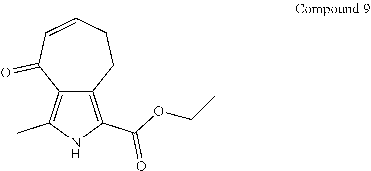 [SMILES: CCOC(=O)c1[nH]c(C)c2c1CCC=CC2=O]

Ethyl 3-methyl-4-oxo-2,4,7,8-tetrahydrocyclohepta[c]pyrrole-1-carboxylate (Compound 9). To a solution of ethyl 4-acetyl-5-methyl-3-(3-oxopropyl)-1H-pyrrole-2-carboxylate (7.30 g, 29.1 mmol, 1 equiv.) in PhMe (291 mL) was added PTSA monohydrate (2.76 g, 14.5 mmol, 0.5 equiv.). The mixture was heated to reflux for 3 h, cooled to rt, evaporated. Concentrated NaHCO₃ solution was added and the aqueous phase was extracted three times with EtOAc. The combined organics were washed with brine, dried over Na₂SO₄ and evaporated. The residue was purified on silica gel column eluting with 20-50% EtOAc in CyH to afford the desired product (3.79 g, 16.2 mmol, 56%) as a white solid. ¹H NMR (400 MHz, CDCl₃) δ 9.32 (s, 1H), 6.66 (dt, J=12.1, 6.0 Hz, 1H), 6.16 (dt, J=12.3, 1.5 Hz, 1H), 4.35 (q, J=7.1 Hz, 2H), 3.33-3.23 (m, 2H), 2.59 (d, J=0.6 Hz, 3H), 2.52 (ddt, J=10.5, 5.9, 1.6 Hz, 2H), 1.39 (t, J=7.1 Hz, 3H); ¹³C NMR (101 MHz, CDCl₃) δ 188.7, 161.5, 143.8, 140.7, 134.4, 132.5, 123.5, 115.9, 60.5, 28.0, 24.4, 14.8, 14.6; HRMS (ESI): calcd. for C₁₃H₁₆NO₃ [M+H]⁺: 234.1125, found: 234.1125.

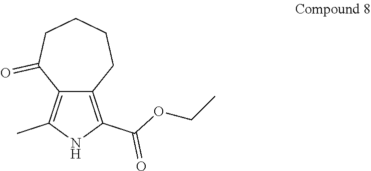 [SMILES: CCOC(=O)c1[nH]c(C)c2c1CCCCC2=O]

Ethyl 3-methyl-4-oxo-2,4,5,6,7,8-hexahydrocyclohepta[c]pyrrole-1-carboxylate (Compound 8). To a solution of ethyl 3-methyl-4-oxo-2,4,7,8-tetrahydrocyclohepta[c]pyrrole-1-carboxylate (3.50 g, 15.0 mmol, 1 equiv.) in EtOAc/MeOH (150 mL, 1:1) under argon was added Pd/C (700 mg, 5% w/w). The reaction was stirred at rt for 16 h under H₂. After filtration and purification on silica gel column (20-60% EtOAc in CyH), to obtain the desired compound as a white solid (3.46 g, 14.7 mmol, 98%). ¹H NMR (400 MHz, CDCl₃) δ 9.62 (s, 1H), 4.32 (q, J=7.1 Hz, 2H), 3.25-3.07 (m, 2H), 2.70-2.57 (m, 2H), 2.51 (s, 3H), 1.89-1.72 (m, 4H), 1.35 (t, J=7.1 Hz, 3H); ¹³C NMR (101 MHz, CDCl₃) δ 200.0, 161.8, 138.7, 134.2, 123.5, 116.6, 60.4, 42.0, 25.3, 23.4, 21.9, 14.5, 13.8; HRMS (ESI): calcd. for C₁₃H₁₆NO₃ [M−H]⁻: 234.1136, found: 234.1136; R_{f}: 0.47 (30% AcOEt in CyH).

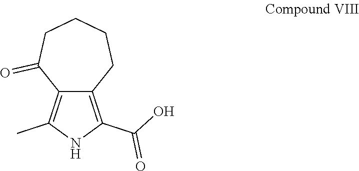

3-methyl-4-oxo-2,4,5,6,7,8-hexahydrocyclohepta[c]pyrrole-1-carboxylic acid (Compound VIII). To a solution of ethyl 3-methyl-4-oxo-2,4,5,6,7,8-hexahydrocyclohepta[c]pyrrole-1-carboxylate (1.15 g, 4.89 mmol, 1 equiv.) was dissolved in EtOH and aqueous 2M NaOH (48.9 mL, 1:1). The mixture was heated at reflux overnight, cooled at 0° C. and neutralized until pH 1 using concentrated aqueous HCl (12M). The resulting precipitate was filtered and recrystallised using CHCl₃ to afford the desired product (723 mg, 3.49 mmol, 71%) as a white solid. ¹H NMR (500 MHz, DMSO-d₆) δ 12.44 (s, 1H), 11.81 (s, 1H), 3.13 (td, J=6.2, 5.3, 2.9 Hz, 2H), 2.55-2.50 (m, 1H), 2.36 (s, 2H), 1.75-1.65 (m, 4H); ¹³C NMR (126 MHz, DMSO-d₆) δ 198.4, 162.3, 138.0, 133.0, 122.3, 116.6, 41.3, 24.8, 22.3, 21.2, 13.1; HRMS (APCI): calcd. for C₁₁H₁₄NO₃ [M+H]⁺: 208.0968, found: 208.0968; R_{f}: 0.63 (100% AcOEt+2% AcOH).

### Example 2. Synthesis of 3-amino-N,N-diethyl-4-methoxybenzenesulfonamide

General Synthetic Scheme

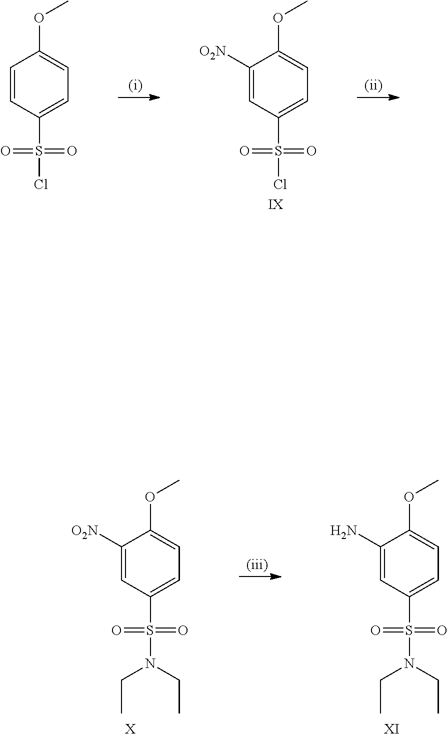

Reaction conditions: i) HNO₃, H₂SO₄, 0° C., 60%; ii) diethylamine (Et₂NH), CH₂Cl₂, 0° C. to rt, 99%; iii) H₂, Pd/C, MeOH/AcOEt, rt, 96%

CH₂Cl₂); R_{f}: 0.38 (60% AcOEt in CyH)

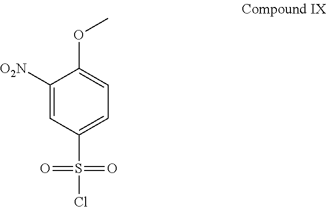

4-methoxy-3-nitrobenzenesulfonyl chloride (Compound IX). To a solution of 4-methoxybenzenesulfonyl chloride (6.18 g, 29.9 mmol, 1 equiv.) in H₂SO₄ (30 mL) at 0° C., was added HNO₃ (1.91 mL, 44.8 mmol, 1.5 equiv.). After 2 h, the solution was poured into ice/water and extracted with Et₂O. The organic phase was washed with water, brine, dried over Na₂SO₄ and evaporated to afford the desired product (4.53 g, 18.0 mmol, 60%) as a white solid. ¹H NMR (500 MHz, CDCl₃) δ 8.51 (d, J=2.5 Hz, 1H), 8.21 (dd, J=9.0, 2.4 Hz, 1H), 7.31 (d, J=9.1 Hz, 1H), 4.11 (s, 3H); ¹³C NMR (126 MHz, CDCl₃) δ 157.7, 139.2, 135.9, 132.8, 125.5, 114.5, 57.6; R_{f}: 0.19 (33% AcOEt in CyH).

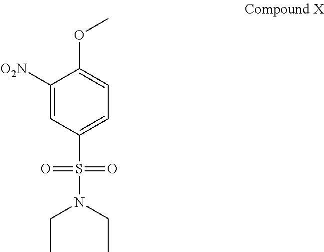 [SMILES: CCN(CC)S(=O)(=O)c1ccc(OC)c([N+](=O)[O-])c1]

N,N-diethyl-4-methoxy-3-nitrobenzenesulfonamide (Compound X). To a solution of 4-methoxy-3-nitrobenzenesulfonyl chloride (4.53 g, 18.0 mmol, 1 equiv.) in CH₂Cl₂ (36 mL) at 0° C., was added diethylamine (9.31 mL, 54.0 mmol, 3 equiv.) dropwise. The solution was then stirred at rt for 16 h, washed with 1M HCl, brine, dried over Na₂SO₄ and evaporated. The residue was purified on silica gel column eluting with 20-80% EtOAc in CyH to afford the desired product (5.15 g, 17.9 mmol, 99%) as a light yellow solid. ¹H NMR (500 MHz, CDCl₃) δ 8.23 (d, J=2.3 Hz, 1H), 7.95 (dd, J=8.8, 2.3 Hz, 1H), 7.19 (d, J=8.9 Hz, 1H), 4.02 (s, 3H), 3.23 (q, J=7.1 Hz, 4H), 1.13 (t, J=7.2 Hz, 6H); ¹³C NMR (126 MHz, CDCl₃) δ 155.4, 139.2, 132.8, 132.7, 124.8, 114.0, 57.1, 42.2, 14.2; HRMS (ESI): calcd. for C₁₁H₁₇N₂O₅ [M+H]⁺: 289.0855, found: 289.0855; R_{f}: 0.40 (100% CH₂Cl₂); R_{f}: 0.38 (60% AcOEt in CyH).

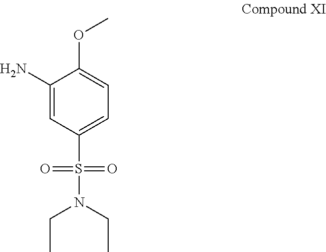 [SMILES: CCN(CC)S(=O)(=O)c1ccc(OC)c(N)c1]

### Example 3. Synthesis of N-(5-(N,N-diethylsulfamoyl)-2-methoxyphenyl)-3-methyl-4-oxo-2,4,5,6,7,8-hexahydrocyclohepta[c]pyrrole-1-carboxamide

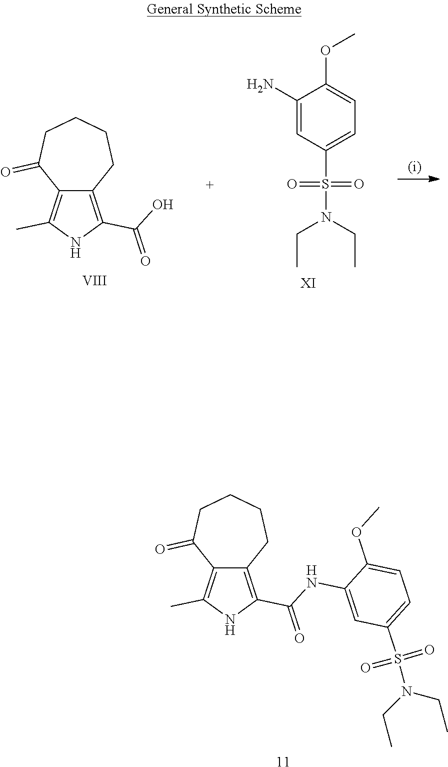

Reaction conditions: i) HCTU, DIEA, DMF, 0° C. to 100° C., 64%

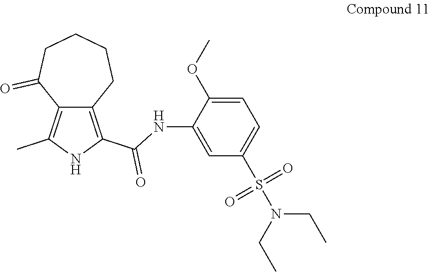 [SMILES: CCN(CC)S(=O)(=O)c1ccc(OC)c(NC(=O)c2[nH]c(C)c3c2CCCCC3=O)c1]

N-(5-(N,N-diethylsulfamoyl)-2-methoxyphenyl)-3-methyl-4-oxo-2,4,5,6,7,8-hexahydrocyclohepta[c]pyrrole-1-carboxamide (Compound 11). To a solution of 3-methyl-4-oxo-2,4,5,6,7,8-hexahydrocyclohepta[c]pyrrole-1-carboxylic acid (100 mg, 0.48 mmol, 1 equiv.) and 3-amino-N,N-diethyl-4-methoxybenzenesulfonamide (155 mg, 0.60 mmol, 1.25 equiv.) in dry DMF (1.9 mL) at −0° C., were added diisopropylethylamine (DIEA) (0.42 mL, 2.41 mmol, 5 equiv.) and HCTU (2-(6-Chloro-1h-benzotriazol-1-yl),1,1,3,3-tetramethylaminium-hexafluorophopsphate) (250 mg, 0.60 mmol, 1.25 equiv.). The reaction was allowed to warm at rt. After 4 h, the mixture was heated at 100° C. for 16 h. Then, DMF was evaporated. Water was added and the aqueous phase was extracted three times with EtOAc. The combined organics were washed with 1H HCl, saturated Na₂CO₃, brine, dried over Na₂SO₄. Purification on silica gel column eluting with 20-100% EtOAc in cyclohexane to afford the desired product (139 mg, 0.31 mmol, 64%) as a light yellow solid. ¹H NMR (400 MHz, CDCl₃) δ 10.13 (s, 1H), 8.84 (d, J=2.3 Hz, 1H), 8.22 (s, 1H), 7.52 (dd, J=8.5, 2.3 Hz, 1H), 6.93 (d, J=8.7 Hz, 1H), 3.98 (s, 3H), 3.22 (q, J=7.1 Hz, 4H), 3.15-3.06 (m, 2H), 2.72-2.62 (m, 2H), 2.49 (d, J=0.5 Hz, 3H), 2.00-1.80 (m, 4H), 1.11 (t, J=7.2 Hz, 6H); ¹³C NMR (101 MHz, CDCl₃) δ 199.5, 159.8, 150.6, 138.6, 132.8, 128.1, 127.9, 123.5, 123.2, 120.7, 118.1, 109.6, 56.5, 42.2, 41.9, 25.8, 24.5, 21.8, 14.3, 13.7; HRMS (ESI): calcd. for C₂₂H₂₉N₃O₅SNa [M+Na]⁺: 470.1720, found: 470.1720; R_{f}: 0.57 (66% AcOEt in CyH).

### Example 4. Synthesis of 2-methoxy-5-(morpholinosulfonyl)aniline

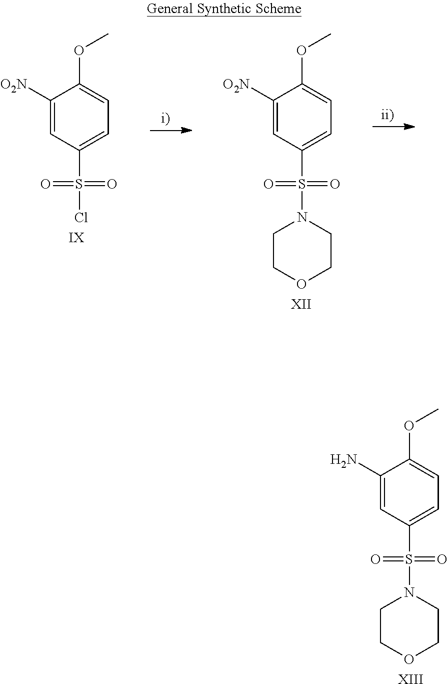

Reaction conditions: i) Morpholine, CH₂Cl₂, 0° C. to rt, 87%; ii) H₂, Pd/C, MeOH/AcOEt, rt, 91%

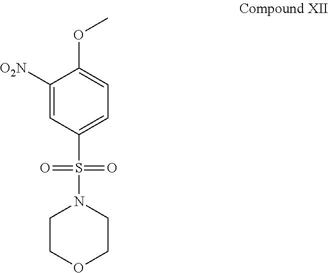 [SMILES: COc1ccc(S(=O)(=O)N2CCOCC2)cc1[N+](=O)[O-]]

4-((4-methoxy-3-nitrophenyl)sulfonyl)morpholine (Compound XII). The compound was synthesized as reported for N,N-diethyl-4-methoxy-3-nitrobenzenesulfonamide (morpholine was used instead of diethylamine), to afford the desired compound (3.14 g, 10.4 mmol, 87%) as a light yellow solid.

¹H NMR (400 MHz, DMSO-d₆) δ 8.20 (d, J=2.3 Hz, 1H), 8.00 (dd, J=8.9, 2.3 Hz, 1H), 7.61 (d, J=9.0 Hz, 1H), 4.04 (s, 3H), 3.68-3.60 (m, 4H), 2.96-2.88 (m, 4H); ¹³C NMR (101 MHz, DMSO) δ 155.2, 138.9, 133.4, 126.3, 124.6, 115.4, 65.2, 57.4, 45.7; HRMS (ESI): calcd. for C₁₁H₁₄N₂O₆SNa [M+Na]⁺: 325.0465, found: 325.0465; R_{f}: 0.35 (60% AcOEt in CyH).

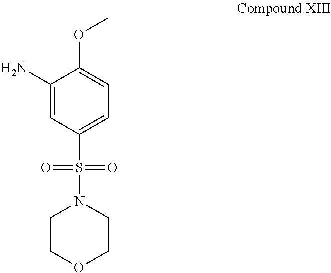 [SMILES: COc1ccc(S(=O)(=O)N2CCOCC2)cc1N]

2-methoxy-5-(morpholinosulfonyl)aniline (Compound XIII). The compound was synthesized as reported for 3-amino-N,N-diethyl-4-methoxybenzenesulfonamide in MeOH/EtOAc (1:1, 50 mL), to afford the desired compound (1.89 g, 6.94 mmol, 91%) as a light yellow solid. ¹H NMR (500 MHz, DMSO-d₆) δ 7.04-6.94 (m, 2H), 6.88 (dt, J=8.4, 2.1 Hz, 1H), 3.84 (d, J=1.6 Hz, 3H), 3.60 (t, J=4.8 Hz, 4H), 2.84-2.74 (m, 4H); ¹³C NMR (126 MHz, DMSO-d₆) δ 149.6, 138.3, 125.8, 116.2, 111.5, 109.9, 65.3, 55.6, 45.9; HRMS (ESI): calcd. for C₁₁H₁₆N₂O₄SNa [M+Na]⁺: 295.0723, found: 295.0723; R_{f}: 0.72 (80% AcOEt in CyH).

### Example 5. Synthesis of N-(2-methoxy-5-(morpholinosulfonyl)phenyl)-3-methyl-4-oxo-2,4,5,6,7,8-hexahydrocyclohepta[c]pyrrole-1-carboxamide

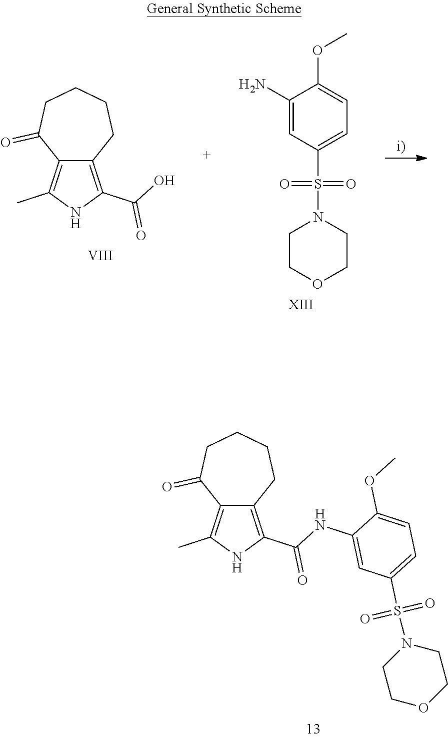

Reaction conditions: i) O-(1H-6-Chlorobenzotriazole-1-yl)-1,1,3,3-tetramethyluronium hexafluorophosphate (HCTU), N,N-Diisopropylethylamine (DIEA), DMF, 0° C. to 100° C., 38%

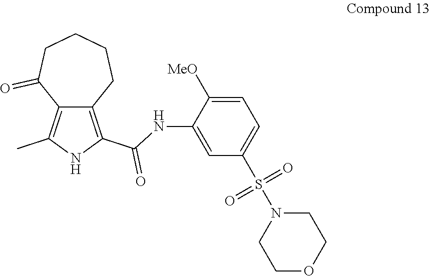 [SMILES: COc1ccc(S(=O)(=O)N2CCOCC2)cc1NC(=O)c1[nH]c(C)c2c1CCCCC2=O]

N-(2-methoxy-5-(morpholinosulfonyl)phenyl)-3-methyl-4-oxo-2,4,5,6,7,8-hexahydrocyclohepta[c]pyrrole-1-carboxamide. The compound was synthesized as reported for N-(5-(N,N-diethylsulfamoyl)-2-methoxyphenyl)-3-methyl-4-oxo-2,4,5,6,7,8-hexahydrocyclohepta[c]-pyrrole-1-carboxamide, to afford the desired compound (84 mg, 0.18 mmol, 38%) as a light yellow solid. ¹H NMR (400 MHz, CDCl₃) δ 9.90 (s, 1H), 8.83 (d, J=2.3 Hz, 1H), 8.25 (s, 1H), 7.48 (dd, J=8.6, 2.3 Hz, 1H), 7.01 (d, J=8.7 Hz, 1H), 4.02 (s, 3H), 3.77-3.65 (m, 4H), 3.13-3.07 (m, 2H), 3.05-2.98 (m, 4H), 2.71-2.66 (m, 2H), 2.50 (s, 3H), 2.01-1.85 (m, 4H); ¹³C NMR (101 MHz, CDCl₃) δ 199.5, 159.8, 151.1, 138.7, 128.3, 127.8, 127.7, 124.0, 123.6, 120.7, 118.9, 109.8, 66.2, 56.6, 46.2, 41.9, 25.9, 24.7, 21.8, 13.8; HRMS (ESI): calcd. for C₂₂H₂₇N₃O₆SNa [M+Na]⁺: 484.1513, found: 484.1513; R_{f}: 0.19 (66% AcOEt in CyH).

### Example 6. Synthesis of 2-methoxy-5-((4-methylpiperazin-1-yl)sulfonyl)aniline

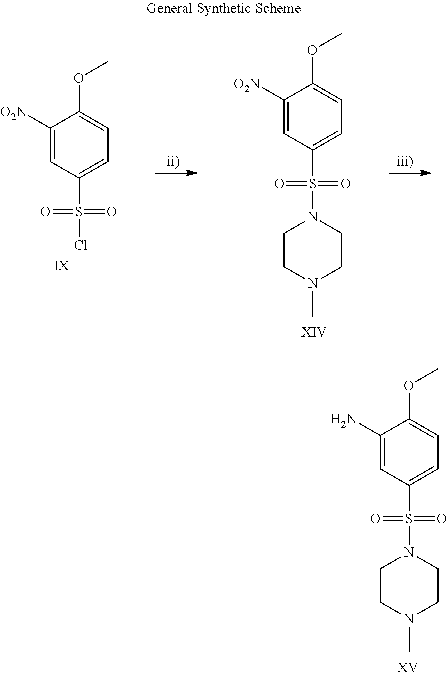

Reaction conditions: i) N-Methylpiperazine, CH₂Cl₂, 0° C. to rt, 87%; ii) H₂, Pd/C, MeOH/AcOEt, rt, 91%

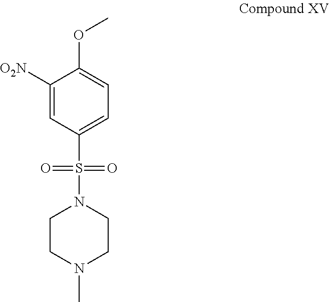 [SMILES: COc1ccc(S(=O)(=O)N2CCN(C)CC2)cc1[N+](=O)[O-]]

2-methoxy-5-((4-methylpiperazin-1-yl)sulfonyl)aniline (Compound XV). The compound was synthesized as reported for 3-amino-N,N-diethyl-4-methoxybenzenesulfonamide in MeOH/EtOAc (1:1, 50 mL), to afford the desired compound (1.66 g, 5.82 mmol, 92%) as a light yellow solid. HRMS (ESI): calcd. for C₁₂H₂₀N₃O₃S [M+H]⁺: 286.1220, found: 286.1220.

### Example 7. Synthesis of N-(2-methoxy-5-((4-methylpiperazin-1-yl)sulfonyl)phenyl)-3-methyl-4-oxo-2,4,5,6,7,8-hexahydrocyclohepta[c]pyrrole-1-carboxamide

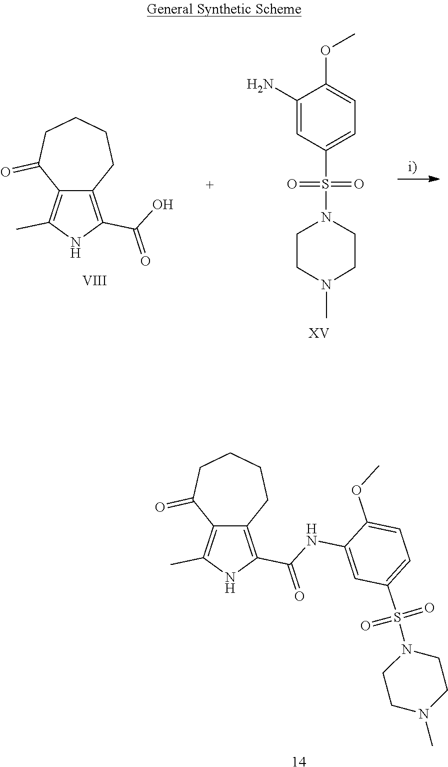

Reaction conditions: i) HCTU, DIEA, DMF, 0° C. to 100° C., 41%

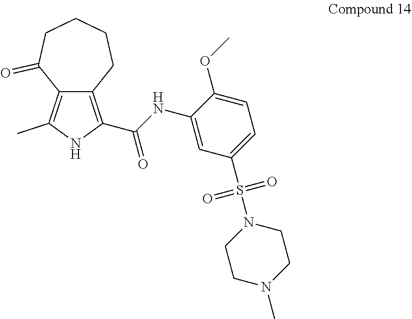 [SMILES: COc1ccc(S(=O)(=O)N2CCN(C)CC2)cc1NC(=O)c1[nH]c(C)c2c1CCCCC2=O]

N-(2-methoxy-5-(4-methylpiperazin-1-yl)sulfonyl)phenyl)-3-methyl-4-oxo-2,4,5,6,7,8-hexahydrocyclohepta[c]pyrrole-1-carboxamide (Compound 14). The compound was synthesized as reported for N-(5-(N,N-diethylsulfamoyl)-2-methoxyphenyl)-3-methyl-4-oxo-2,4,5,6,7,8-hexahydrocyclohepta[c]pyrrole-1-carboxamide, to afford the desired compound (94 mg, 0.20 mmol, 41%) as a light yellow solid. ¹H NMR (400 MHz, CDCl₃) δ 9.98 (s, 1H), 8.83 (d, J=2.3 Hz, 1H), 8.23 (s, 1H), 7.47 (dd, J=8.6, 2.3 Hz, 1H), 6.98 (d, J=8.7 Hz, 1H), 4.00 (s, 3H), 3.14-2.99 (m, 6H), 2.73-2.62 (m, 2H), 2.49 (d, J=0.4 Hz, 3H), 2.45 (t, J=5.0 Hz, 4H), 2.24 (s, 3H), 2.00-1.81 (m, 4H); ¹³C NMR (101 MHz, CDCl₃) δ 199.5, 159.7, 151.0, 138.6, 128.3, 127.9, 127.7, 123.9, 123.6, 120.8, 118.8, 109.7, 56.6, 54.2, 46.1, 45.8, 41.9, 25.9, 24.6, 21.8, 13.8; HRMS (ESI): calcd. for C₂₃H₃₁N₄O₅S [M+H]⁺: 475.2010, found: 475.2010; R_{f}: 0.05 (66% AcOEt in CyH); R_{f}: 0.36 (12% MeOH in CH₂Cl₂+0.1% NEt₃).

### Example 8. Synthesis of 5-(azepan-1-ylsulfonyl)-2-methoxyaniline

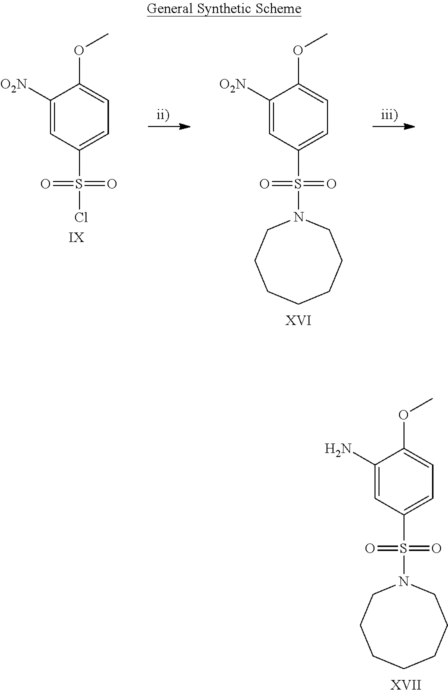

Reaction conditions: i) Azepane, CH₂Cl₂, 0° C. to rt, 94%; ii) H₂, Pd/C, MeOH/AcOEt, rt, 95%

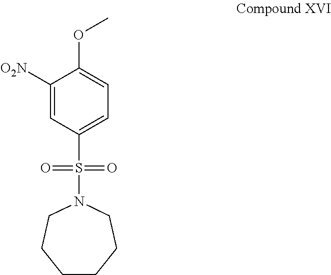 [SMILES: COc1ccc(S(=O)(=O)N2CCCCCC2)cc1[N+](=O)[O-]]

1-((4-methoxy-3-nitrophenyl)sulfonyl)azepane (Compound XVI). The compound was synthesized as reported for N,N-diethyl-4-methoxy-3-nitrobenzenesulfonamide (azepane was used instead of diethylamine), to afford the desired compound (3.52 g, 11.2 mmol, 94%) as a light yellow solid. ¹H NMR (500 MHz, CDCl₃) δ 8.23 (d, J=2.3 Hz, 1H), 7.95 (dd, J=8.8, 2.3 Hz, 1H), 7.19 (d, J=8.9 Hz, 1H), 4.03 (s, 3H), 3.32-3.19 (m, 4H), 1.72 (ddddd, J=9.9, 8.1, 6.7, 3.3, 2.2 Hz, 4H), 1.63-1.53 (m, 4H); ¹³C NMR (126 MHz, CDCl₃) δ 155.4, 139.3, 132.6, 132.0, 124.7, 113.9, 57.1, 48.4, 29.2, 26.9; HRMS (ESI): calcd. for C₁₃H₁₉N₂O₅S [M+H]⁺: 315.1009, found: 315.1009; R_{f}: 0.56 (60% AcOEt in CyH).

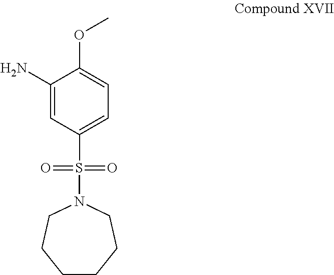 [SMILES: COc1ccc(S(=O)(=O)N2CCCCCC2)cc1N]

5-(azepan-1-ylsulfonyl)-2-methoxyaniline (Compound XVII). The compound was synthesized as reported for 3-amino-N,N-diethyl-4-methoxybenzenesulfonamide in MeOH/EtOAc (1:1, 50 mL), to afford the desired compound (1.72 g, 6.05 mmol, 95%) as a light yellow solid. ¹H NMR (400 MHz, DMSO-d₆) δ 8.85 (s, 3H), 7.68 (d, J=2.3 Hz, 1H), 7.50 (dd, J=8.6, 2.4 Hz, 1H), 7.22 (dd, J=8.7, 1.1 Hz, 1H), 3.91 (s, 3H), 3.24-3.12 (m, 4H), 1.72-1.42 (m, 8H); ¹³C NMR (101 MHz, DMSO-d₆) δ 153.2, 131.0, 127.0, 123.8, 118.8, 111.8, 56.3, 47.6, 28.4, 26.3; HRMS (ESI): calcd. for C₁₃H₂₁N₂O₃S [M+H]⁺: 285.1287, found: 285.1287; R_{f}: 0.48 (40% AcOEt in PE).

### Example 9. Synthesis of N-(5-(azepan-1-ylsulfonyl)-2-methoxyphenyl)-3-methyl-4-oxo-2,4,5,6,7,8-hexahydrocyclohepta[c]pyrrole-1-carboxamide

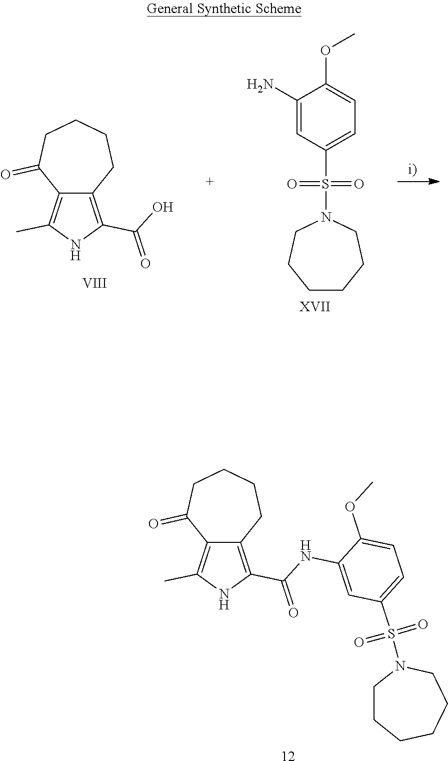

Reaction conditions: i) HCTU, DIEA, DMF, 0° C. to 100° C., 73%

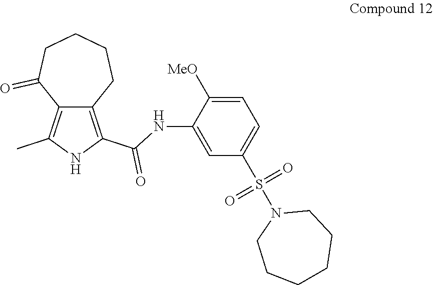 [SMILES: COc1ccc(S(=O)(=O)N2CCCCCC2)cc1NC(=O)c1[nH]c(C)c2c1CCCCC2=O]

N-(5-(azepan-1-ylsulfonyl)-2-methoxyphenyl)-3-methyl-4-oxo-2,4,5,6,7,8-hexahydrocyclohepta[c]pyrrole-1-carboxamide (Compound 12). The compound was synthesized as reported for N-(5-(N,N-diethylsulfamoyl)-2-methoxyphenyl)-3-methyl-4-oxo-2,4,5,6,7,8-hexahydrocyclohepta[c]pyrrole-1-carboxamide, to afford the desired compound (167 mg, 0.35 mmol, 73%) as a light yellow solid. ¹H NMR (400 MHz, CDCl₃) δ 10.07 (s, 1H), 8.83 (d, J=2.3 Hz, 1H), 8.23 (s, 1H), 7.51 (dd, J=8.6, 2.3 Hz, 1H), 6.94 (d, J=8.6 Hz, 1H), 3.99 (s, 3H), 3.31-3.23 (m, 4H), 3.12-3.07 (m, 2H), 2.72-2.63 (m, 2H), 2.49 (d, J=0.5 Hz, 3H), 1.99-1.82 (m, 4H), 1.68 (dtt, J=10.9, 4.7, 2.4 Hz, 4H), 1.61-1.52 (m, 4H); ¹³C NMR (101 MHz, CDCl₃) δ 199.5, 159.8, 150.6, 138.6, 132.1, 128.1, 127.9, 123.5, 123.1, 120.8, 117.9, 109.6, 56.5, 48.4, 41.9, 29.2, 27.0, 25.8, 24.5, 21.8, 13.7; HRMS (ESI): calcd. for C₂₄H₃₁N₃O₅SNa [M+Na]⁺: 496.1877, found: 496.1877; R_{f}: 0.40 (66% AcOEt in CyH).

### Example 10—Synthesis of Compound 23

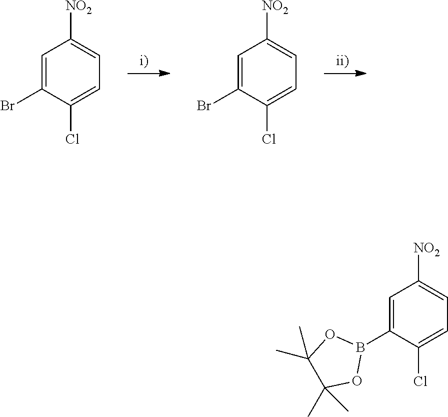 [SMILES: O=[N+]([O-])c1ccc(Cl)c(Br)c1 ; O=[N+]([O-])c1ccc(Cl)c(Br)c1 ; CC1(C)OB(c2cc([N+](=O)[O-])ccc2Cl)OC1(C)C]

### 2-Bromo-1-chloro-4-nitrobenzene

NBS (14.1 g, 79.2 mmol, 1.2equiv) were added portionwise to a solution of 4-chloronitrobenzene (10.4 g, 66.0 mmol, 1.0 equiv) in conc. H₂SO₄ (30 mL over 45 min at 60° C. After 2 h the reaction mixture was poured on ice. The precipitated solids were filtered off, washed first with H₂O and then with pentane. The remaining solid was dried under reduced pressure to obtain the desired product as a yellowish solid (11.3 g, 47.7 mmol, 72%). Mp: 53-54° C.

¹H-NMR (400 MHz, CDCl₃): δ=7.63 (d, J=8.8 Hz, 1H), 8.12 (dd, J=8.8 Hz, J=2.6 Hz, 1H), 8.50 (d, J=2.6 Hz, 1H). ¹³C-NMR (101 MHz, CDCl₃): δ=123.3, 123.4, 127.5, 128.9, 130.9, 141.9.

### 2-(2-Chloro-5-nitrophenyl)-4,4,5,5-tetramethyl-1,3,2-dioxaborolane

A suspension of 2-(3,5-dinitrophenyl)-4,4,5,5-tetramethyl-1,3,2-dioxaborolane (100 mg, 423 mmol, 1.0 equiv), KOAc (81 mg, 825 mmol, 2.0 equiv.), B₂(pin)₂ (120 mg, 465 mmol, 1.1 equiv.), Pd(dppf)Cl₂ (9.3 mg, 12.7 mmol, 3 mol %) in degassed dioxane (1.2 mL) and DMSO (20 μL) was stirred overnight at 90° C. The solvent was removed under reduced pressure and an aqueous solution of NaOH (2M) was added, the mixture was stirred for 10 min, filtered and washed two times with Et₂O. The aqueous phase was acidified with conc. HCl and afterwards three times extracted with EA. The organic phase was dried over Na₂SO₄ and evaporated to obtain the title compound as a yellowish solid (63.1 mg, 0.222 mmol, 52%). Mp: 89-92° C.

¹H-NMR (500 MHz, CDCl₃): δ=1.39 (s, 14H), 7.51 (d, J=8.8 Hz, 1H), 8.18 (dd, J=8.8, 2.8 Hz, 1H), 8.55 (d, J=2.9 Hz, 1H). ¹³C-NMR (101 MHz, CDCl₃): δ=25.0, 85.1, 126.6, 130.6, 131.5, 132.6, 146.1, 146.7. HRMS (neg. APCI): [M+O₂]⁻ calc. for C₁₂H₁₅NO₆BCl: 315.0686; found: 315.0688.

### 1-bromo-3-methyl-5,6,7,8-tetrahydrocyclohepta[c]pyrrol-4(2H)-one

General synthetic scheme

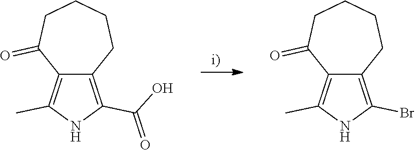

Pyridine (0.54 mL, 6.76 mmol, 5 equiv.) followed by pyridinium tribromide (648 mg, 2.03 mmol, 1.5 equiv.) were added to a solution of 3-methyl-4-oxo-2,4,5,6,7,8-hexahydrocyclohepta[c]pyrrole-1-carboxylic acid (280 mg, 1.35 mmol, 1 equiv.) in CH₂Cl₂ (13.5 mL) at 0° C. The mixture was allowed to return to room temperature and stirred overnight. Aqueous hydrochloric acid (HCl, 0.5M) was added and the organic phase was collected, dried over Na₂SO₄ and evaporated. Purification on flash chromatography column afford the desired product (159 mg, 0.66 mmol, 49%) as a colourless solid, which decomposes at room temperature.

¹H NMR (400 MHz, CDCl₃) δ 9.67 (s, 1H), 2.71-2.64 (m, 4H), 2.49 (d, J=0.5 Hz, 3H), 1.92-1.70 (m, 4H); ¹³C NMR (101 MHz, CDCl₃) δ 200.1, 137.2, 123.9, 121.1, 96.0, 41.8, 25.1, 24.2, 22.3, 13.9, HRMS (ESI): calcd. for C₁₀H₁₃NOBr [M+H]⁺: 242.0175, found: 242.0175; R_{f}: 0.45 (33% AcOEt in cyclohexane).

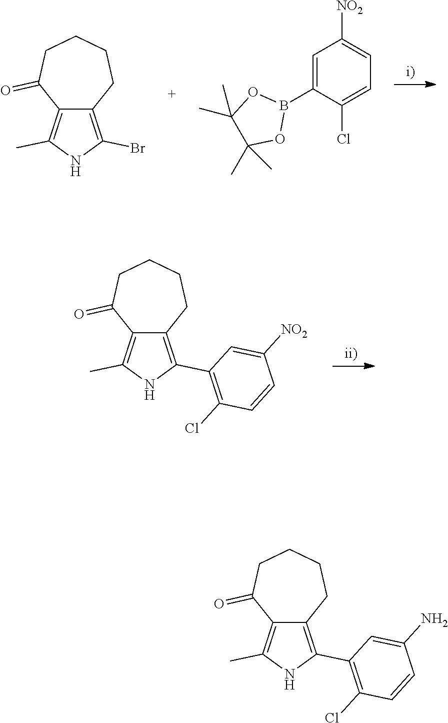 [SMILES: Cc1[nH]c(Br)c2c1C(=O)CCCC2 ; CC1(C)OB(c2cc([N+](=O)[O-])ccc2Cl)OC1(C)C ; Cc1[nH]c(-c2cc([N+](=O)[O-])ccc2Cl)c2c1C(=O)CCCC2 ; Cc1[nH]c(-c2cc(N)ccc2Cl)c2c1C(=O)CCCC2]

### 1-(2-Chloro-5-nitrophenyl)-3-methyl-5,6,7,8-tetrahydrocyclohepta[c]pyrrol-4(2H)-one

A mixture of 1-bromo-3-methyl-5,6,7,8-tetrahydrocyclohepta[c]pyrrol-4(2H)-one (73 mg, 0.30 mmol, 1.0 equiv.), 2-(2-Chloro-5-nitrophenyl)-4,4,5,5-tetramethyl-1,3,2-dioxaborolane (0.13 g, 0.45 mmol, 1.5 equiv.), 1,3,5,7-tetramethyl-6-phenyl-2,4,8-trioxa-6-phosphaadamantane (18 mg, 0.06 mmol, 0.02 equiv.), Pd₂dba₃ (14 mg, 15 μmol, 0.05 equiv.), K₃PO₄ (0.19 g, 0.90 mmol, 3.0 equiv.) in degassed dioxane/H₂O (1.5 mL) was stirred 3.75 h at 70° C., then filtered over celite and evaporated. The residue was purified by column chromatography (10-85% EA in PE) to afford the desired product (34 mg, 0.11 mmol, 35%) as a light yellow solid. Mp: 198-199° C.

¹H-NMR (500 MHz, CDCl₃): δ=1.80-1.95 (m, 4H), 2.58 (s, 3H), 2.67-2.73 (m, 4H), 7.64 (d, J=8.8 Hz, 1H), 8.12 (d, J=8.8 Hz, J=2.7 Hz, 1H), 8.19 (d, J=2.7 Hz, 1H), 8.38 (br. s, 1H). ¹³C-NMR (126 MHz, CDCl₃): δ=14.0, 22.2, 23.9, 25.6, 42.0, 121.0, 122.0, 123.3, 125.7, 126.9, 131.3, 132.7, 136.2, 140.2, 146.6, 200.1. HRMS (pos. ESI): [M+H]⁺ calc. for C₁₆H₁₅O₃N₂Cl: 319.0844; found: 319.0842.

### 1-(5-Amino-2-chlorophenyl)-3-methyl-5,6,7,8-tetrahydrocyclohepta[c]pyrrol-4(2M-one (Compound 23)

To a solution of 1-(2-chloro-5-nitrophenyl)-3-methyl-5,6,7,8-tetrahydrocyclohepta[c]pyrrol-4(2H)-one (33 mg, 0.11 mmol, 1.0 equiv.) and NH₄Cl (12 mg, 0.23 mmol, 2.0 equiv.) in EtOH/H₂O (4:1, 2.3 mL) Fe powder (22 mg, 0.40 mmol, 3.5 equiv.) was added. The mixture was stirred under reflux for 4 h, afterwards filtered first over cotton, then celite. EtOH was evaporated, water was added and the aqueous phase was three times extracted with EA. The combined organic phases were washed with brine, dried over Na₂SO₄ and evaporated. The residue was purified by reversed phase column chromatography (MeCN/H₂O) to afford the desired product as a colourless solid (23 mg, 0.79 mmol, 69%). Mp: 169-171° C.

¹H-NMR (500 MHz, CDCl₃): δ=1.79-1.92 (m, 4H), 2.54 (d, J=0.5 Hz, 3H), 2.63-2.75 (m, 4H), 3.60-3.83 (m, 2H), 6.54-6.62 (m, 2H), 7.18-7.23 (m, 1H), 8.27 (s, 1H). ¹³C-NMR (126 MHz, CDCl₃): δ=13.9, 22.2, 23.8, 25.7, 42.0, 115.7, 118.3, 121.5, 122.5, 123.4, 123.6, 130.8, 131.5, 134.7, 145.2, 220.3. HRMS (pos. ESI): [M+H]⁺ calc. for C₁₆H₁₈ON₂Cl: 289.1102; found: 289.1101.

### Example 11—Synthesis of Compound (24)

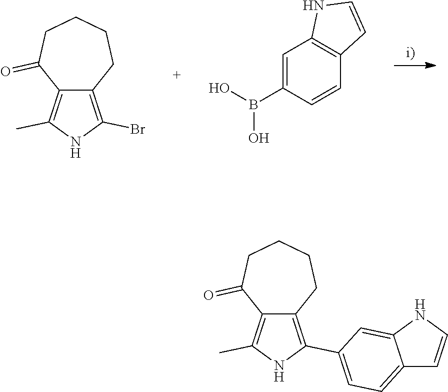 [SMILES: Cc1[nH]c(Br)c2c1C(=O)CCCC2 ; OB(O)c1ccc2cc[nH]c2c1 ; Cc1[nH]c(-c2ccc3cc[nH]c3c2)c2c1C(=O)CCCC2]

### 1-(1H-indol-6-yl)-3-methyl-5,6,7,8-tetrahydrocyclohepta[c]pyrrol-4(2H)-one (Compound 24)

To 1-bromo-3-methyl-5,6,7,8-tetrahydrocyclohepta[c]pyrrol-4(2H)-one (50 mg, 0.21 mmol, 1 equiv.), (1H-indol-6-yl)boronic acid (50 mg, 0.31 mmol, 1.5 equiv.), 1,3,5,7-tetramethyl-6-phenyl-2,4,8-trioxa-6-phosphaadamantane (24 mg, 0.02 mmol, 0.1 equiv.), Pd₂dba₃ (19 mg, 0.02 mmol, 0.1 equiv.), K₃PO₄ (131 mg, 0.62 mmol, 3 equiv.) degassed dioxane/H₂O (2.1 mL) under argon was added. The mixture was stirred at 60° C. for 16 h, then filtered on celite and evaporated. The mixture was purified on silica gel column eluting with 30-70% EtOAc in PE to afford the desired product (37 mg, 0.13 mmol, 64%) as a light yellow solid.

¹H NMR (500 MHz, MeOD-d₄) δ 7.58 (dd, J=8.2, 0.7 Hz, 1H), 7.45-7.36 (m, 1H), 7.20 (d, J=3.1 Hz, 1H), 7.08 (dd, J=8.2, 1.5 Hz, 1H), 6.45 (dd, J=3.2, 0.9 Hz, 1H), 2.90-2.84 (m, 2H), 2.67-2.62 (m, 2H), 2.49 (s, 3H), 1.90-1.72 (m, 4H); ¹³C NMR (126 MHz, MeOD-d₄) δ 202.4, 136.7, 135.9, 128.9, 127.2, 126.4, 125.5, 121.9, 121.2, 120.7, 120.1, 110.7, 101.9, 41.8, 26.3, 23.8, 22.7, 13.5; R_{f}: 0.25 (40% AcOEt in cyclohexane).

### Example 12—Synthesis of Compounds (26) and (27)

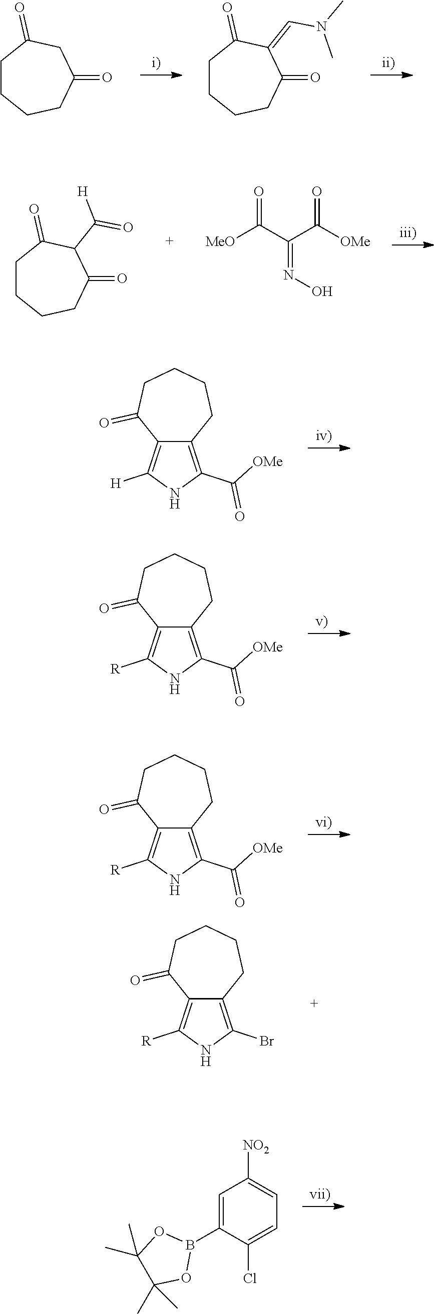

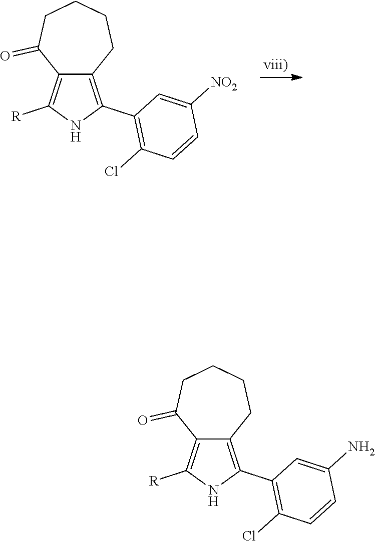

### 2-((Dimethylamino)methylene)cycloheptane-1,3-dione (Step I)

To a solution of cycloheptane-1,3-dione (7.61 g, 60.3 mmol, 1.0 equiv.) in dry DCM (86 mL) N,N-dimethylformamide dimethyl acetal (10.5 mL, 9.34 g, 78.4 mmol, 1.3 equiv.) was added. The mixture was stirred at room temperature overnight, evaporated and purified by column chromatography (8% MeOH in DCM) to afford the desired product (9.49 g, 52.4 mmol, 87%) as a yellow solid. Mp: 91-92° C.

¹H-NMR (400 MHz, CDCl₃): δ=1.83 (m_{c}, 4H), 2.56 (m_{c}, 4H), 2.78 (s, 3H), 3.27 (s, 3H), 7.69 (s, 1H). ¹³C-NMR (101 MHz, CDCl₃): δ=22.3, 40.6, 43.3, 48.0, 112.9, 159.6, 200.1

### 2,7-Dioxacycloheptane-1-carbaldehyde (Step II)

To a solution of 2-((dimethylamino)methylene)-cycloheptane-1,3-dione (9.49 g, 52.3 mmol, 1.0 equiv.) in THF (70 mL) aqueous HCl (1%, 260 mL) was added. The solution was stirred for 3 h at room temperature, THF was removed and the aqueous phase was extracted three times with EA. The combined organic phases were washed with water, brine, dried over Na₂SO₄ and evaporated to afford the desired product (7.52 g, 48.8 mmol, 93%) as a light orange oil. ¹H-NMR (500 MHz, CDCl₃): δ=1.86-1.95 (m, 4H), 2.64-2.70 (m, 2H), 2.74-2.78 (m, 2H), 9.29 (br. s, 1H). ¹³C-NMR (126 MHz, CDCl₃): δ=21.2, 22.1, 36.2, 41.1, 117.3, 190.3, 198.4, 199.2. HRMS (neg. APCI): [M−H]⁻ calc. for C₈H₉O₃: 153.0557; found: 153.0558.

### Methyl 4-oxo-2,4,5,6,7,8-hexahydrocyclohepta[c]pyrrole-1-carboxylate (Step III)

Dimethyl 2-(hydroxy-imino)malonate (10.2 g, 63.5 mmol, 1.35 equiv), 2,7-dioxocycloheptane-1-carbaldehyde (7.25 g, 47.0 mmol, 1.0 equiv) and NaOAc (13.5 g, 165 mmol, 3.5 equiv.) were dissolved in AcOH (157 mL) and heated to 90° C. Zn (12.3 g, 188 mmol, 4.0 equiv) was added portionwise. The reaction mixture was stirred for 5 h at 95° C., poured on ice and the aqueous phase was extracted three times with EA. The combined organic layers were washed twice with NaOH, then brine, dried over Na₂SO₄ and evaporated. The residue was purified by column chromatography (33-40% EA in PE) to afford the desired product as a colourless solid (2.64 g, 12.8 mmol, 27%). Mp: 129-132° C.

¹H-NMR (400 MHz, CDCl₃): δ=1.80-1.97 (m, 4H), 2.69 (m_{c}, 2H), 3.19 (m_{c}, 2H), 3.87 (s, 3H), 7.50 (d, J=3.5 Hz, 1H), 9.62 (br. s, 1H). ¹³C-NMR (101 MHz, CDCl₃): δ=22.2, 24.4, 25.9, 42.2, 51.6, 119.6, 126.0, 127.9, 132.9, 161.9, 199.2. HRMS (pos. APCI): [M+H]⁺ calc. for C₁₁H₁₃NO₃: 208.0968; found: 208.0969.

### Methyl 4-oxo-3-propyl-2,4,5,6,7,8-hexahydrocyclohepta[c]pyrrole-1-carboxylate and Methyl 3-butyl-4-oxo-2,4,5,6,7,8-hexahydrocyclohepta[c]pyrrole-1-carboxylate (Step IV)

Methyl 4-oxo-2,4,5,6,7,8-hexahydrocyclohepta[c]pyrrole-1-carboxylate (0.95 g, 4.6 mmol, 1.0 equiv), 1-bromopropane (0.83 mL, 1.1 g, 9.1 mmol, 2.0 equiv), norbornene (0.86 g, 9.1 mmol, 2.0 equiv), KHCO₃ (1.4 g, 14 mmol, 3.0 equiv) and PdCl₂(MeCN)₂ (87 mg, 0.34 mmol, 0.01 equiv.) were stirred in DMA (3.4 mL) and heated to 90° C. overnight. After cooling to room temperature the mixture was filtered through a pad of celite, which was rinsed three times with Et₂O. The filtrate was washed with H₂O and the aqueous phase was extracted two times with Et₂O. The combined organic layers were washed with brine, dried over Na₂SO₄ and evaporated. The residue was purified by column chromatography (25% EA in PE) to afford the desired as a yellow solid (0.75 g, 3.0 mmol, 66%). Mp: 125-127° C.,

¹H-NMR (400 MHz, CDCl₃): δ=0.97 (t, J=7.3 Hz, 3H), 1.60-1.75 (m, 2H), 1.80-1.91 (m, 4H), 2.62-2.70 (m, 2H), 2.86-2.95 (m, 2H), 3.13-3.23 (m, 1H), 3.86 (s, 3H), 8.85 (s, 1H)¹³C-NMR (101 MHz, CDCl₃): δ=14.0, 22.0, 22.0, 23.4, 25.3, 29.7, 42.2, 51.5, 77.5, 116.5, 123.3, 134.4, 142.8, 161.9, 199.8 HRMS (pos. ESI): [M+H]⁺ calc. for C₁₄H₂₀NO₃: 250.1438; found: 250.1438.

For methyl 3-butyl-4-oxo-2,4,5,6,7,8-hexahydrocyclohepta[c]pyrrole-1-carboxylate 1-bromobutane (0.97 mL, 1.2 g, 9.1 mmol, 2.0 equiv) was used instead of 1-bromopropane. The desired product was obtained as a yellow solid (0.78 g, 3.0 mmol, 59%). Mp: 123-125° C., ¹H-NMR (500 MHz, CDCl₃): δ=0.93 (t, J=7.3 Hz, 3H), 1.38 (dq, J=14.7, 7.3 Hz, 2H), 1.55-1.66 (m, 2H), 1.79-1.88 (m, 4H), 2.61-2.69 (m, 2H), 2.88-2.95 (m, 2H), 3.14-3.21 (m, 2H), 3.86 (s, 3H), 8.97 (s, 1H). ¹³C-NMR (126 MHz, CDCl₃): δ=14.0, 21.9, 22.7, 23.3, 25.3, 27.5, 30.9, 42.1, 51.5, 116.5, 123.1, 134.5, 143.1, 162.0, 199.9. HRMS (pos. ESI): [M+H]⁺ calc. for C₁₅H₂₂NO₃: 264.1594; found: 264.1596.

### 4-Oxo-3-propyl-2,4,5,6,7,8-hexahydrocyclohepta[c]pyrrole-1-carboxylic acid and (Step V)

LiOH.H₂O (1.3 g, 30 mmol, 10 equiv) was added to a solution of methyl 4-oxo-3-propyl-2,4,5,6,7,8-hexahydrocyclohepta[c]pyrrole-1-carboxylate (0.75 g, 3.0 mmol, 1.0 equiv) in a mixture of THF/H₂O/MeOH (30 mL, 1:1:1). The reaction mixture was stirred at rt overnight. The organic solvents were evaporated and the aqueous phase was acidified with conc. HCl, extracted three times with EA, dried over Na₂SO₄ and concentrated in vacuo to obtain the desired product as a colourless solid (0.69 g, 2.9 mmol, 97%). Mp: 182-187° C.,

¹H-NMR (500 MHz, Acetone-d₆): δ=0.90 (t, J=7.4 Hz, 3H), 1.59-1.69 (m, 2H), 1.77-1.85 (m, 4H), 2.54-2.64 (m, 2H), 2.85-2.96 (m, 2H), 3.18-3.29 (m, 2H), 10.77 (s, 1H). ¹³C-NMR (126 MHz, Acetone-d₆): δ=14.1, 22.5, 23.4, 26.0, 29.5, 42.4, 117.3, 123.5, 134.5, 143.4, 162.4, 199.0. HRMS (neg. APCI): [M−H]⁻ calc. for C₁₃H₁₆NO₃: 234.1136; found: 234.1130.

### 4-Oxo-3-butyl-2,4,5,6,7,8-hexahydrocyclohepta[c]pyrrole-1-carboxylic acid (Step V)

LiOH.H₂O (1.2 g, 30 mmol, 10 equiv) was added to a solution of methyl 4-oxo-3-butyl-2,4,5,6,7,8-hexahydrocyclohepta[c]pyrrole-1-carboxylate (0.78 g, 3.0 mmol, 1.0 equiv) in a mixture of THF/H₂O/MeOH (30 mL, 1:1:1). The reaction mixture was stirred at rt overnight. The organic solvents were evaporated and the aqueous phase was acidified with conc. HCl, extracted three times with EA, dried over Na₂SO₄ and concentrated in vacuo to obtain the desired product as a colourless solid (0.20 g, 0.79 mmol, 27%). Mp: 134-139° C.

¹H-NMR (500 MHz, Acetone-d₆): δ=0.90 (t, J=7.4 Hz, 3H), 1.28-1.39 (m, 2H), 1.55-1.64 (m, 2H), 1.76-1.85 (m, 4H), 2.55-2.62 (m, 2H), 2.90-2.99 (m, 2H), 3.20-3.27 (m, 2H), 10.79 (s, 1H). ¹³C-NMR (126 MHz, Acetone-d₆): δ=14.1, 22.5, 23.2, 23.4, 26.0, 27.4, 32.4, 42.4, 117.5, 123.4, 134.4, 143.5, 162.8, 199.0. HRMS (neg. APCI): [M+H]+ calc. for C₁₄H₁₈NO₃: 248.1292; found: 248.1291.

### 1-Bromo-3-propyl-5,6,7,8-tetrahydrocyclohepta[c]pyrrol-4(2H)-one

Pyridine (1.0 mL, 0.98 g, 13 mmol, 5.0 equiv) and PyHBr₃ (1.2 g, 3.6 mmol, 1.5 equiv) were added to a solution of 4-oxo-3-propyl-2,4,5,6,7,8-hexahydrocyclohepta[c]pyrrole-1-carboxylic acid (0.57 g, 2.4 mmol, 1.0 equiv) in DCM (24 mL) at 0° C. The reaction mixture was stirred at rt for 4.5 h. Aqueous HCl (0.5 M) was added and the phases were separated. The organic phase was dried over Na₂SO₄ and concentrated in vacuo. The residue was purified via column chromatography (5-9% EA in pentane) to obtain the desired product as a colourless solid which turns black and decomposes at rt (0.43 g, 1.6 mmol, 66%). The product was used without further characterisation due to it's instability. HRMS (pos. ESI): [M+H]⁺ calc. for C₁₂H₁₇NOBr: 270.0488; found: 270.0489.

### 1-Bromo-3-butyl-5,6,7,8-tetrahydrocyclohepta[c]pyrrol-4(2H)-one

Pyridine (0.28 mL, 0.27 g, 3.4 mmol, 5.0 equiv) and PyHBr₃ (0.33 g, 1.0 mmol, 1.5 equiv) were added to a solution of 4-oxo-3-butyl-2,4,5,6,7,8-hexahydrocyclohepta[c]pyrrole-1-carboxylic acid (0.17 g, 0.68 mmol, 1.0 equiv) in DCM (7.0 mL) at 0° C. The reaction mixture was stirred at rt for 4.5 h. Aqueous HCl (0.5 M) was added and the phases were separated. The organic phase was dried over Na₂SO₄ and concentrated in vacuo. The residue was purified via column chromatography (5-6% EA in pentane) to obtain the desired product as a colourless solid which turns black and decomposes at rt (79 mg, 0.28 mmol, 41%). The product was used without further characterisation due to it's instability. HRMS (pos. ESI): [M+H]⁺ calc. for C₁₃H₁₉NOBr: 284.0645; found: 284.0643.

### 1-(2-Chloro-5-nitrophenyl)-3-propyl-5,6,7,8-tetrahydrocyclohepta[c]pyrrol-4(2H)-one

To 1-bromo-3-propyl-5,6,7,8-tetrahydrocyclohepta[c]pyrrol-4(2H)-one (100 mg, 0.37 mmol, 1.0 equiv.), 2-(2-Chloro-5-nitrophenyl)-4,4,5,5-tetramethyl-1,3,2-dioxaborolane (0.16 g, 0.56 mmol, 1.5 equiv.), 1,3,5,7-tetramethyl-6-phenyl-2,4,8-trioxa-6-phosphaadamantane (22 mg, 74 μmol, 0.02 equiv.), Pd₂dba₃ (17 mg, 19 μmol, 0.05 equiv.), K₃PO₄ (0.24 g, 1.1 mmol, 3.0 equiv.) in degassed dioxane/H₂O (1.9 mL) was stirred 2.5 h at 70° C., then filtered over celite and evaporated. The residue was purified by column chromatography (3-85% EA in PE) to afford the desired product (25 mg, 73 μmol, 20%) as a light yellow solid. Mp: 68-70° C.

¹H-NMR (500 MHz, CDCl₃): δ=0.98 (t, J=7.4, 7.4 Hz, 3H), 1.61-1.75 (m, 2H), 1.77-1.93 (m, 4H), 2.64-2.72 (m, 4H), 2.89-2.99 (m, 2H), 7.63 (d, J=8.8 Hz, 1H), 8.10 (dd, J=8.8, 2.7 Hz, 1H), 8.19 (d, J=2.7 Hz, 1H), 8.65 (s, 1H). ¹³C-NMR (126 MHz, CDCl₃): δ=14.0, 22.1, 22.4, 23.8, 25.5, 29.6, 42.0, 121.1, 121.4, 123.2, 125.6, 127.0, 131.3, 132.7, 140.3, 140.8, 146.6, 200.0. HRMS (pos. ESI): [M+H]⁺ calc. for C₁₈H₂₀N₂O₃Cl: 347.1157; found: 347.1160.

### 1-(2-Chloro-5-nitrophenyl)-3-butyl-5,6,7,8-tetrahydrocyclohepta[c]pyrrol-4(2H)-one

A mixture of 1-bromo-3-butyl-5,6,7,8-tetrahydrocyclohepta[c]pyrrol-4(2H)-one (0.8 g, 0.28 mmol, 1.0 equiv.), 2-(2-Chloro-5-nitrophenyl)-4,4,5,5-tetramethyl-1,3,2-dioxaborolane (0.12 g, 0.42 mmol, 1.5 equiv.), 1,3,5,7-tetramethyl-6-phenyl-2,4,8-trioxa-6-phosphaadamantane (16 mg, 56 μmol, 0.02 equiv.), Pd₂dba₃ (13 mg, 14 μmol, 0.05 equiv.), K₃PO₄ (0.18 g, 0.85 mmol, 3.0 equiv.) in degassed dioxane/H₂O (1.4 mL) was stirred 2.5 h at 70° C., then filtered over celite and evaporated. The residue was purified by reversed phase column chromatography (MeCN/H₂O) to afford the desired product (12 mg, 33 μmol, 12%) as a yellow oil.

¹H-NMR (500 MHz, CDCl₃): δ=0.94 (t, J=7.3 Hz, 3H), 1.41 (dq, J=14.7, 7.4 Hz, 2H), 1.61-1.69 (m, 2H), 1.81-1.95 (m, 4H), 2.69 (ddd, J=7.6, 4.4, 1.8 Hz, 4H), 2.94-3.03 (m, 2H), 7.64 (d, J=8.8 Hz, 1H), 8.11 (dd, J=8.8, 2.7 Hz, 1H), 8.19 (d, J=2.7 Hz, 1H), 8.44 (s, 1H). ¹³C-NMR (126 MHz, CDCl₃): δ=14.1, 22.1, 22.7, 23.8, 25.6, 27.4, 31.2, 42.1, 121.0, 121.5, 123.2, 125.7, 126.9, 131.3, 132.7, 140.2, 140.9, 146.6, 199.9. HRMS (neg. ESI): [M−H]⁻ calc. for C₁₉H₂₀N₂O₃Cl: 359.1168; found: 359.1165.

### 1-(5-Amino-2-chlorophenyl)-3-propyl-5,6,7,8-tetrahydrocyclohepta[c]pyrrol-4(2H)-one (Compound 26)

To a solution of 1-(2-chloro-5-nitrophenyl)-3-propyl-5,6,7,8-tetrahydrocyclohepta[c]pyrrol-4(2H)-one (25 mg, 72 μmol, 1.0 equiv.) and NH₄Cl (7.7 mg, 0.14 mmol, 2.0 equiv.) in EtOH/H₂O (4:1, 1.4 mL) Fe powder (14 mg, 0.25 mmol, 3.5 equiv.) was added. The mixture was stirred under reflux for 4 h, afterwards filtered first over cotton, then celite. EtOH was evaporated, water was added and the aqueous phase was three times extracted with EA. The combined organic phases were washed with brine, dried over Na₂SO₄ and evaporated. The residue was purified by reversed phase column chromatography (MeCN/H₂O) to afford the desired product as a yellow oil (15 mg, 47 μmol, 66%).

¹H-NMR (500 MHz, CDCl₃): δ=0.98 (t, J=7.3 Hz, 4H), 1.63-1.73 (m, 2H), 1.76-1.92 (m, 4H), 2.64-2.68 (m, 2H), 2.68-2.72 (m, 2H), 2.90-2.97 (m, 2H), 6.58-6.63 (m, 2H), 7.18-7.23 (m, 1H), 8.37 (s, 1H). ¹³C-NMR (126 MHz, CDCl₃): δ=14.1, 22.3, 22.3, 23.7, 25.7, 29.7, 42.1, 115.8, 118.4, 121.0, 122.5, 123.5, 123.7, 130.8, 131.6, 139.3, 145.2, 200.2. HRMS (pos. ESI): [M+H]⁺ calc. for C₁₈H₂₂N₂OCl: 317.1415; found: 317.1422.

1-(5-Amino-2-chlorophenyl)-3-butyl-5,6,7,8-tetrahydrocyclohepta[c]pyrrol-4(2H)-one (Compound 27). To a solution of 1-(2-chloro-5-nitrophenyl)-3-butyl-5,6,7,8-tetrahydrocyclohepta[c]pyrrol-4(2H)-one (12 mg, 33 μmol, 1.0 equiv.) and NH₄Cl (3.5 mg, 66 μmol, 2.0 equiv.) in EtOH/H₂O (4:1, 0.67 mL) Fe powder (6.5 mg, 0.12 mmol, 3.5 equiv.) was added. The mixture was stirred under reflux for 4 h, afterwards filtered first over cotton, then celite. EtOH was evaporated, water was added and the aqueous phase was three times extracted with EA. The combined organic phases were washed with brine, dried over Na₂SO₄ and evaporated. The residue was purified by reversed phase column chromatography (MeCN/H₂O) to afford the desired product as a yellow oil (6.6 mg, 20 μmol, 60%). ¹H-NMR (500 MHz, CDCl₃): δ=0.93 (t, J=7.3 Hz, 3H), 1.40 (h, J=7.4 Hz, 2H), 1.58-1.69 (m, 2H), 1.76-1.92 (m, 4H), 2.63-2.74 (m, 4H), 2.93-3.00 (m, 2H), 6.56-6.68 (m, 2H), 7.21 (d, J=8.3 Hz, 1H), 8.30 (s, 1H). ¹³C-NMR (126 MHz, CDCl₃): δ=14.1, 22.3, 22.7, 23.8, 25.7, 27.5, 31.2, 42.1, 115.8, 118.5, 120.9, 122.6, 123.5, 123.7, 130.9, 131.6, 139.6, 145.1, 200.2. HRMS (pos. ESI): [M+H]⁺ calc. for C₁₉H₂₄N₂OCl: 331.1572; found: 331.1576.

Protein Preparation, Crystallization and Structure Determination

BRD4-BD1 was expressed and purified as described previously (Filippakopoulos et al, (2012), Cell 149(1): 241-231) with the exception of the final buffer for crystallization and ITC analysis (20 mM Hepes/NaOH pH 7.5, 150 mM NaCl). Crystals were grown in the presence of 3.5 M Na-Formate (pH 7.5) at a protein concentration of 10 mg/ml and a ligand concentration of 2 mM added directly to the protein prior to crystallization from a 100 mM stock solution in DMSO. Data were collected at 100K using either a Rigaku HF-007 rotating anode X-ray generator equipped with VariMaxHF optics and a Saturn944 CCD detector or a mar345 image plate respectively at λ=1.54179 Å or at the PXI beamline at the Swiss Light Source at λ=1.000 Å with a Pilatus detector. Data processing and reduction was done with iMOSFLM (Leslie AGW PH (2007), Evolving Methods for Macromolecular Crystallography 245, 41.51), POINTLESS, and SCALA (Kabsch W (2010) Xds. Acta crystallographica. Section D, Biological crystallography 66(Pt 2):125-132; Bruker (2008) SADABS, SAINT and XPREP (Bruker AXS Inc., Madison, Wis., USA); Collaborative Computational Project N (1994) The CCP4 suite: programs for protein crystallography, Acta crystallographica, Section D, Biological crystallography 50(Pt 5):760-763), or with XDS, XSCALE, XDSCONV (Evans P (2006), Scaling and assessment of data quality, Acta crystallographica, Section D, Biological crystallography 62(Pt 1):72-82), and XPREP (Evans P R (2011), An introduction to data reduction: space-group determination, scaling and intensity statistics, Acta crystallographica, Section D, Biological crystallography 67(Pt 4):282-292).

BRD4.ligand complexes crystallized in space group P2₁2₁2₁. The structures were solved by molecular replacement with PHASER (McCoy A J, et al. (2007), Phaser crystallographic software, Journal of applied crystallography 40(Pt 4):658-674) with apoBRD4 as search model (internal data) yielding one molecule per asymmetric unit. Compounds were modelled into 2Fo-Fc electron density maps using AFITT-CL (version 2.1.0, OpenEye Scientific Software, Inc., Santa Fe, NM, USA.) Model building and real space refinement was done with COOT (Murshudov G N, Vagin A A, & Dodson E J (1997), Refinement of macromolecular structures by the maximum-likelihood method, Acta crystallographica. Section D, Biological crystallography 53(Pt 3):240-255) reciprocal space refinement against the calculated data was done with Refmac5, as implemented in the CCP4 suite (Murshudov G N, et al. (2011), REFMAC5 for the refinement of macromolecular crystal structures, Acta crystallographica. Section D, Biological crystallography 67(Pt 4):355-367; Vaguine A A, Richelle J, & Wodak S J (1999), SFCHECK: a unified set of procedures for evaluating the quality of macromolecular structure-factor data and their agreement with the atomic model, Acta crystallographica, Section D, Biological crystallography 55(Pt 1):191-205). Final structure validation was done with procheck/sfcheck (Wlodek S, Skillman A G, & Nicholls A (2006), Automated ligand placement and refinement with a combined force field and shape potential, Acta crystallographica, Section D, Biological crystallography 62(Pt 7):741-749).

Isothermal Titration Calorimetry

ITC experiments for the determination of the dissociation constant K_{d} were done with a Microcal VP-ITC microcalorimeter (GE Healthcare) at 25° C. using ligand concentrations between 10 and 50 μM in the sample cell and BRD4 concentrations between 120 and 600 μM in the injection syringe. Data were obtained in discrete titration experiments with an injection volume of 12 μl per injection. Subsequently, the heats per injection were calculated as integrals and after normalization against the molar concentrations plotted against the molar ratio as implemented in Microcal Origin. Finally, data were fitted according to a single set of sites binding model with

$$Q = {nM}_{t} \times Δ {HV}_{0} \times \frac{1}{2} \times ( 1 + \frac{{X}_{t}}{{nM}_{t}} + \frac{1}{{nKM}_{t}} - \sqrt{{( 1 + \frac{{X}_{t}}{{nM}_{t}} + \frac{1}{{nKM}_{t}} )}^{2} - \frac{4 {X}_{t}}{{nM}_{t}}} ) \qquad ( 1 )$$

as function to calculate the overall sum of heat of the titration and with

$$Δ Q ( i ) = Q ( i ) + \frac{{dV}_{i}}{{V}_{0}} \times ( \frac{Q ( i ) + Q ( i - 1 )}{2} ) - Q ( i - 1 ) \qquad ( 2 )$$

(According to Microcal's manual “ITC Data Analysis in Origin” (September 1998)) as function describing the sum of heat of each individual injection. A correction term was included to compensate for displacement of volume of the sample cell in the course of subsequent injections according to the manufacturer's manual “ITC Data Analysis in Origin” (Microcal).

The dissociation constant K_{d}, is commonly used to describe the affinity between a ligand L and a protein, i.e. how tightly a ligand binds to a particular protein. The dissociation constant has molar units which correspond to the concentration of ligand at which the binding site on a particular protein is half occupied, i.e. the concentration of the ligand at which the concentration of protein with ligand bound equals the concentration of protein with no ligand bound. The smaller the dissociation constant, the higher the affinity between ligand and protein.

Table 1 shows the dissociation constants K_{D} for two bromodomains (BRD4(1) and BRPF1B) as well as the concentration of the respective compound required to reduce the population of a culture of HL-60 cells by 50% (GI₅₀)

TABLE 1

| Cpd. of | K_{D} BRD4(1) | K_{D} BRPF1B | GI50_{HL60} |
|---|---|---|---|
| formula | (nM) | (nM) | (nM) |
| 1″ | 6410 | 5080 | n.d. |
| 8 | 4562 | 1050 | n.d. |
| 9 | 8281 | 2050 | n.d. |
| 11 | 143.9 | n.d. | 1360 |
| 12 | 176.7 | n.d. | 570 |
| 13 | 77.8 | n.d. | 170 |
| 14 | 259.6 | n.d. | 2080 |
| 15 | 47.3 | n.d. | n.d. |
| 16 | 417.2 | n.d. | n.d. |
| 18 | 72.2 | n.d. | n.d. |
| 19 | n.d. | 910 | n.d. |
| 20 | n.d. | 567 | n.d. |
| 21 | n.d. | 1990 | n.d. |
| 22 | n.d. | 2310 | n.d. |
| 23 | n.d. | 166 | n.d. |
| 24 | n.d. | 639 | n.d. |
| 25 | n.d. | 713 | n.d. |
| 26 | 6900 | 7000 | n.d. |
| 27 | >10000 | >10000 | |

(n.d. = not determined)

Bromodomain Profiling

Bromodomain profiling was carried out on the basis of BROMOscan™. This platform accounted for the indirect determination of the dissociation constants between 19 bromodomains and the compound of formula (13), by binding competition against a reference immobilized ligand.

The overall structure of the complex of the compound of formula (13) and BRD4-BD1 revealed the already well-characterized bromodomain fold with a bundle of four α-helices, interconnected by three loops of different length. The termini of the helices bundle are flanked by elongational loops, which tightly pack against the protein core producing a compact and rather rigid structure.

The compound of formula (13) is bound in a pocket located at the end of the longitudinal axis running through the helix bundle which points towards the N-terminus. Consequently, it occupies the same pocket as the native K_{ac} substrate.

Moreover, it mimics the K_{ac} interaction with BRD4-BD1 by positioning the 4-acyl substitution in the pyrrole ring toward the highly conserved Asn140, thus engaging in hydrogen bond interactions with Asn140 and the equally conserved water molecule that bridges to the conserved Tyr97. The pyrrole ring is located deep in the recognition pocket, and complements the hydrophobic pocket defined by the four conserved waters with a 5-methyl substitution. The surface complementarity between the ligand and the recognition pocket is further achieved by the 3-ethyl substitution in the pyrrole ring. The presence of the heteroatom in the core of the compound of formula (I″) allows for a key hydrogen-bond donor interaction with the proline's backbone in position 82. Such interaction has not been described previously, and may have an important role in fixating the compound in the recognition site.

Apart from this major determinant of ligand recognition by BRD4-BD1, the compound of formula (13) also explores another patch of interactions.

The phenylsulphonamide moiety is placed along the ZA channel, such that a T-shaped CH-π interaction with Trp81 is established, whereby a perfect orthogonal orientation of both aromatic systems is created with Trp81 directly pointing towards the centre of the phenyl moiety of the compound of formula (13). Such interactions have already been reported in other drug-protein interactions. In addition, Leu92 serves as lid from the opposing side and together with Trp81 it forms a special configuration which will be referred to hereinafter as WL trap.

Compounds that bind the bromodomain prevent its binding to the immobilized ligand thus reducing the amount of protein captured. Conversely, molecules that do not bind the bromodomain have no effect on the amount of protein captured. Hits are identified by measuring the amount of bromodomain captured in test versus control samples.

Protocol Description

Bromodomain assays for Kd determination: T7 phage strains displaying bromodomains were grown in parallel in 24-well blocks in an E. coli host derived from the BL21 strain. E. coli were grown to log-phase and infected with T7 phage from a frozen stock (multiplicity of infection=0.4) and incubated with shaking at 32° C. until lysis (90-150 minutes). The lysates were centrifuged (5,000×g) and filtered (0.2 μm) to remove cell debris. Streptavidin-coated magnetic beads were treated with biotinylated small molecule or acetylated peptide ligands for 30 minutes at room temperature to generate affinity resins for bromodomain assays. The liganded beads were blocked with excess biotin and washed with blocking buffer (SeaBlock (Pierce), 1% BSA, 0.05% Tween 20, 1 mM DTT) to remove unbound ligand and to reduce non-specific phage binding. Binding reactions were assembled by combining bromodomains, liganded affinity beads, and test compounds in 1× binding buffer (17% SeaBlock, 0.33×PBS, 0.04% Tween 20, 0.02% BSA, 0.004% Sodium azide, 7.4 mM DTT). Test compounds were prepared as 1000× stocks in 100% DMSO. Kds were determined using an 11-point 3-fold compound dilution series with one DMSO control point. All compounds for Kd measurements are distributed by acoustic transfer (non-contact dispensing) in 100% DMSO. The compounds were then diluted directly into the assays such that the final concentration of DMSO was 0.09%. All reactions performed in polypropylene 384-well plates. Each was a final volume of 0.02 ml. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 2 μM non-biotinylated affinity ligand) and incubated at room temperature with shaking for 30 minutes. The bromodomain concentration in the eluates was measured by qPCR.

Compound Handling for Kd determination: An 11-point 3-fold serial dilution of each test compound was prepared in 100% DMSO at 1000× final test concentration. All compounds for Kd measurements are distributed by acoustic transfer (non-contact dispensing) in 100% DMSO. The compounds were then diluted directly into the assays such that the final concentration of DMSO was 0.09%. Most Kds were determined using a compound top concentration=10,000 nM. If the initial Kd determined was <0.169 nM (the lowest concentration tested), the measurement was repeated with a serial dilution starting at a lower top concentration.

Binding Constants (Kds): Binding constants (Kds) were calculated with a standard dose-response curve using the Hill equation:

$$Response = \frac{Background + Signal - Background}{1 + ( {Kd}^{Hill Slope} \text{/} {Dose}^{Hill Slope} )}$$

The Hill Slope was set to −1. Curves were fitted using a non-linear least square fit with the Levenberg-Marquardt algorithm.

The results are given in Table 2 and Table 3.

TABLE 2

| | Compound | | | | | | |
|---|---|---|---|---|---|---|---|
| | 23 | 11 | 12 | 13 | 14 | 15 | 16 |
| Bromodomain- | Kd | Kd | Kd | Kd | Kd | Kd | Kd |
| (Gene symbol) | (nM) | (nM) | (nM) | (nM) | (nM) | (nM) | (nM) |
| ATAD2A | >10000 | n.d. | n.d. | n.d. | n.d. | n.d. | n.d. |
| ATAD2B | >10000 | n.d. | n.d. | n.d. | n.d. | n.d. | n.d. |
| BAZ2A | 1900 | n.d. | n.d. | n.d. | n.d. | n.d. | n.d. |
| BAZ2B | 1800 | n.d. | n.d. | n.d. | n.d. | n.d. | n.d. |
| BRD1 | 3100 | n.d. | n.d. | n.d. | n.d. | n.d. | n.d. |
| BRD2(1) | 130 | n.d. | n.d. | 45 | n.d. | n.d. | n.d. |
| BRD2(1, 2) | 84 | n.d. | n.d. | n.d. | n.d. | n.d. | n.d. |
| BRD2(2) | 200 | n.d. | n.d. | 290 | n.d. | n.d. | n.d. |
| BRD3(1) | 170 | n.d. | n.d. | 100 | n.d. | n.d. | n.d. |
| BRD3(1, 2) | 100 | n.d. | n.d. | n.d. | n.d. | n.d. | n.d. |
| BRD3(2) | 330 | n.d. | n.d. | 140 | n.d. | n.d. | n.d. |
| BRD4(1) | 250 | 85 | 170 | 60 | 300 | 82 | 150 |
| BRD4(1, 2) | 160 | n.d. | n.d. | n.d. | n.d. | n.d. | n.d. |
| BRD4(2) | 270 | n.d. | n.d. | 220 | n.d. | n.d. | n.d. |
| BRD4(short-iso.) | 180 | n.d. | n.d. | n.d. | n.d. | n.d. | n.d. |
| BRD7 | 430 | 250 | 310 | 85 | 160 | 440 | 5.5 |
| BRD8(1) | 2600 | n.d. | n.d. | n.d. | n.d. | n.d. | n.d. |
| BRD8(2) | >10000 | n.d. | n.d. | n.d. | n.d. | n.d. | n.d. |
| BRD9 | 67 | 160 | 530 | 67 | 68 | 300 | 4.3 |
| BRDT(1) | 240 | n.d. | n.d. | 670 | n.d. | n.d. | n.d. |
| BRDT(1, 2) | 420 | n.d. | n.d. | n.d. | n.d. | n.d. | n.d. |
| BRDT(2) | 620 | n.d. | n.d. | 1000 | n.d. | n.d. | n.d. |
| BRPF1 | 130 | 420 | 2500 | 520 | 320 | 1800 | 1300 |
| BRPF3 | 6000 | n.d. | n.d. | n.d. | n.d. | n.d. | n.d. |
| CECR2 | 970 | 1100 | 8100 | n.d. | 240 | 580 | 760 |
| CREBBP | 420 | n.d. | n.d. | n.d. | n.d. | n.d. | n.d. |
| EP300 | 1200 | 2100 | 9000 | n.d. | 1800 | 3500 | 990 |
| FALZ | 2600 | n.d. | n.d. | n.d. | n.d. | n.d. | n.d. |
| GCN5L2 | >10000 | n.d. | n.d. | n.d. | n.d. | n.d. | n.d. |
| PBRM1(2) | 4200 | n.d. | n.d. | n.d. | n.d. | n.d. | n.d. |
| PBRM1(5) | 3800 | n.d. | n.d. | n.d. | n.d. | n.d. | n.d. |
| PCAF | >10000 | n.d. | n.d. | n.d. | n.d. | n.d. | n.d. |
| SMARCA2 | >10000 | n.d. | n.d. | n.d. | n.d. | n.d. | n.d. |
| SMARCA4 | >10000 | n.d. | n.d. | n.d. | n.d. | n.d. | n.d. |
| TAF1(2) | 4100 | n.d. | n.d. | n.d. | n.d. | n.d. | n.d. |
| TAF1L(2) | 6800 | n.d. | n.d. | n.d. | n.d. | n.d. | n.d. |
| TRIM24 | 1400 | n.d. | n.d. | n.d. | n.d. | n.d. | n.d. |
| (Bromo.) | | | | | | | |
| TRIM24(PHD, | 5300 | n.d. | n.d. | n.d. | n.d. | n.d. | n.d. |
| Bromo.) | | | | | | | |
| TRIM33(PHD, | 7700 | n.d. | n.d. | n.d. | n.d. | n.d. | n.d. |
| Bromo.) | | | | | | | |
| WRD9(2) | >10000 | n.d. | n.d. | n.d. | n.d. | n.d. | n.d. |

TABLE 3
(Results for compounds 23, 26 and 27)

| | Compound | | |
|---|---|---|---|
| Bromodomain | 23 | 26 | 27 |
| (Gene Symbol) | Kd (nM) | Kd (nM) | Kd (nm) |
| BRD4(1) | 250 | 6900 | >10000 |
| BRD7 | 430 | 1100 | >10000 |
| BRD9 | 67 | 150 | 800 |
| BRDT(1) | 240 | 6100 | >10000 |
| BRPF1 | 130 | 7000 | >10000 |
| CECR2 | 970 | >10000 | >10000 |

The results show that the compounds of formula (26) and (27) have a higher selectivity for certain bromodomains evaluated compared to the compound of formula (23) which, in turn, shows a very good activity as inhibitor (as evidenced by low values for Kd) for all bromodomains evaluated in Table 3.

Bromodomain assays for % Ctrl determination: T7 phage strains displaying bromodomains were grown in parallel in 24-well blocks in an E. coli host derived from the BL21 strain. E. coli were grown to log-phase and infected with T7 phage from a frozen stock (multiplicity of infection=0.4) and incubated with shaking at 32° C. until lysis (90-150 minutes). The lysates were centrifuged (5,000×g) and filtered (0.2 μm) to remove cell debris. Streptavidin-coated magnetic beads were treated with biotinylated small molecule or acetylated peptide ligands for 30 minutes at room temperature to generate affinity resins for bromodomain assays. The liganded beads were blocked with excess biotin and washed with blocking buffer (SeaBlock (Pierce), 1% BSA, 0.05% Tween 20, 1 mM DTT) to remove unbound ligand and to reduce non-specific phage binding. Binding reactions were assembled by combining bromodomains, liganded affinity beads, and test compounds in 1× binding buffer (16% SeaBlock, 0.32×PBS, 0.02% BSA, 0.04% Tween 20, 0.004% Sodium azide, 7.9 mM DTT). Test compounds were prepared as 1000× stocks in 100% DMSO and subsequently diluted 1:25 in monoethylene glycol (MEG). The compounds were then diluted directly into the assays such that the final concentrations of DMSO and MEG were 0.1% and 2.4%, respectively. All reactions were performed in polypropylene 384-well plates in a final volume of 0.02 ml. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 2 μM non-biotinylated affinity ligand) and incubated at room temperature with shaking for 30 minutes. The bromodomain concentration in the eluates was measured by qPCR.

The compounds were screened at a concentration of 10 000 nM and results for primary screen binding are reported as % of control where lower numbers indicate a stronger binding

The % Ctrl values were determined as follows:

% Ctrl=[(test compound signal-positive control signal)/(negative control signal-positive control signal)]×100

where test compound was the compound of formula (13), negative control was DMSO and positive control was the control compound

Table 3 shows the results for a group of 32 bromodomains:

TABLE 3

| Target bromodomain | % Ctrl @ 10 000 nM |
|---|---|
| ATAD2A | 94 |
| ATAD2B | 97 |
| BAZ2A | 24 |
| BAZ2B | 6.8 |
| BRD1 | 26 |
| BRD2(1) | 0 |
| BRD2(2) | 0.1 |
| BRD3(1) | 0.05 |
| BRD3(2) | 0 |
| BRD4(1) | 0 |
| BRD4(2) | 0 |
| BRD7 | 0 |
| BRD9 | 0 |
| BRDT(1) | 0 |
| BRDT(2) | 0.65 |
| BRPF1 | 0.15 |
| BRPF3 | 35 |
| CECR2 | 1.3 |
| CREBBP | 4.7 |
| EP300 | 1.4 |
| FALZ | 6.8 |
| GCN5L2 | 44 |
| PBRM1(2) | 75 |
| PBRM1(5) | 90 |
| PCAF | 20 |
| SMARCA2 | 83 |
| SMARCA4 | 71 |
| TAF1(2) | 7.4 |
| TAF1L(2) | 40 |
| TRIM24(PHD, Bromo) | 82 |
| TRIM33(PHD, Bromo) | 60 |
| WDR9(2) | 81 |

The results show that the compounds showed very good to average binding affinity to a variety of bromodomains (less than 35% Ctrl).

The results indicate that the compounds in accordance with the present invention provide a promising starting point for the development of novel potent bromodomain inhibitors.

Plasmodium falciparum Inhibitor Treatment for 72 Hours (One Replication Cycle)

P. falciparum cultures were treated for 72 hours with 100 μm of compounds (19) to (22), (24) and (25) or DMSO as control. The cultures were stained for DNA content with fluorescent dye Hoechst 33342 (20 μm) and for RNA content with thiazole orange (1 μm). The double negative population represented uninfected erythrocytes (devoid of DNA and low in RNA content) whereas the P. falciparum infected erythrocytes were gated as early ring, mid trophozoite and late schizont stages based on their DNA and RNA content. The degree of parasitemia was determined by flow cytometry after 72 hours of drug treatment and the compounds in accordance with the present invention yielded a lower degree of parasitemia than the control with the compound of formula (24) being particularly effective in this regard in each of the stages (early ring, mid trophozoite and schizont).

1. A compound of formula (1) or formula (2) 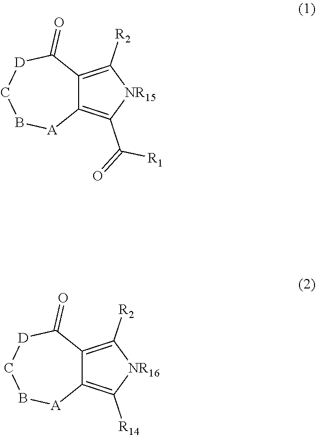
wherein
A, B, C and D, which may be the same or different, independent of one another represent —C(R7Rs)-, —C(R9R10)-C(R11R12)-, —N(R13)-, -0- or —S— with the proviso that the total number of ring atoms of the ring comprising ring members A, B, C and D is 7 to 9,
R1 is selected from the group consisting of OH, OR3, NH2, NHR4 and NRsR6 R2 is a substituted or unsubstituted alkyl group with 1 to 8 carbon atoms or a substituted or unsubstituted alkoxy group with 1 to 8 carbon atoms in which one or more of the hydrogen atoms may be optionally replaced by halogen,
R3 and R4, independent of each other, represent an alkyl group with 1 to 6 carbon atoms or a substituted or unsubstituted alkylaryl, aryl or heteroaryl group,
Rs and R6, independent of each other, represent an alkyl group with 1 to 6 carbon atoms or a substituted or unsubstituted aryl or heteroaryl group or form together with the nitrogen atom to which they are attached, a six- or seven membered, substituted or unsubstituted heterocyclic ring,
R7 to R13, independent of each other, represent hydrogen, hydroxyl, substituted or unsubstituted alkyl groups with 1 to 4 carbon atoms, substituted or unsubstituted alkoxy groups with 1 to 4 carbon atoms and substituted or unsubstituted hydroxyalkyl groups with 1 to 4 carbon atoms or wherein two of R7 to R13 located at adjacent atoms, form together a chemical bond or a ring,
R14 represents a substituted or unsubstituted aryl or heteroaryl group, and
R1s and R16, independent of each other, are hydrogen or a C1 to C4-alkyl group.

2. The compound of formula (1) or (2) in accordance with claim 1 wherein A, B, C and D and Care —(CR?Rs)-.

3. The compound of formula (1) or (2) in accordance with claim 2 wherein R2 is an alkyl group or a haloalkyl group with 1 to 4 carbon atoms.

4. The compound of formula (1) in accordance with claim 3 wherein R1 is a group NHR4 wherein R4 is as defined in claim 1.

5. The compound of formula (1) in accordance with claim 4 wherein R4 is a substituted or unsubstituted aryl group, in particular a substituted or unsubstituted phenyl group.

6. The compound of formula (1) in accordance with claim 5 wherein R4 is represented by formula (3) 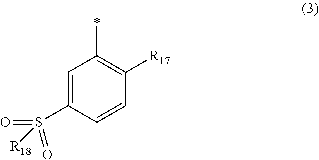
wherein * represents the site of attachment to the nitrogen atom,
R11 is hydroxyl or an alkoxy group with 1 to 4 carbon atoms and R1s is NR19R2o wherein R19 and R2o, independent of each other, represent an alkyl group with 1 to 4 carbon atoms, a substituted or unsubstituted aryl ring or together with the nitrogen atom to which they are attached form a substituted or unsubstituted heterocyclic ring with 4 to 7 ring atoms.

7. The compound of formula (1) in accordance with claim 3 wherein R1 is a group —OR3 wherein R3 is as defined in claim 1.

8. The compound of formula (1) in accordance with claim 4 represented by formula (1″)

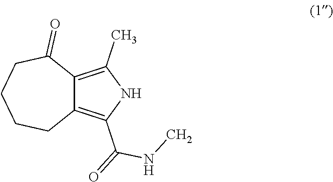 [SMILES: CNC(=O)c1[nH]c(C)c2c1CCCCC2=O]

9. The compound of formula (2) in accordance with claim 3 wherein R14 is a substituted or unsubstituted phenyl group or a substituted or unsubstituted oxadiazole group.

10. The compound of formula (2) in accordance with claim 9 wherein R2 is an alkyl group with 1 to 4 carbon atoms.

11. The compound of formula (2) in accordance with claim 10 wherein R14 is a substituted or unsubstituted phenyl or oxadiazole group.

12. The compound of formula (2) in accordance with claim 11 wherein R16 is hydrogen.

13. The compound of formula (1) in accordance with claim 6 represented by formula (4), formula (5), formula (6) or formula (7) 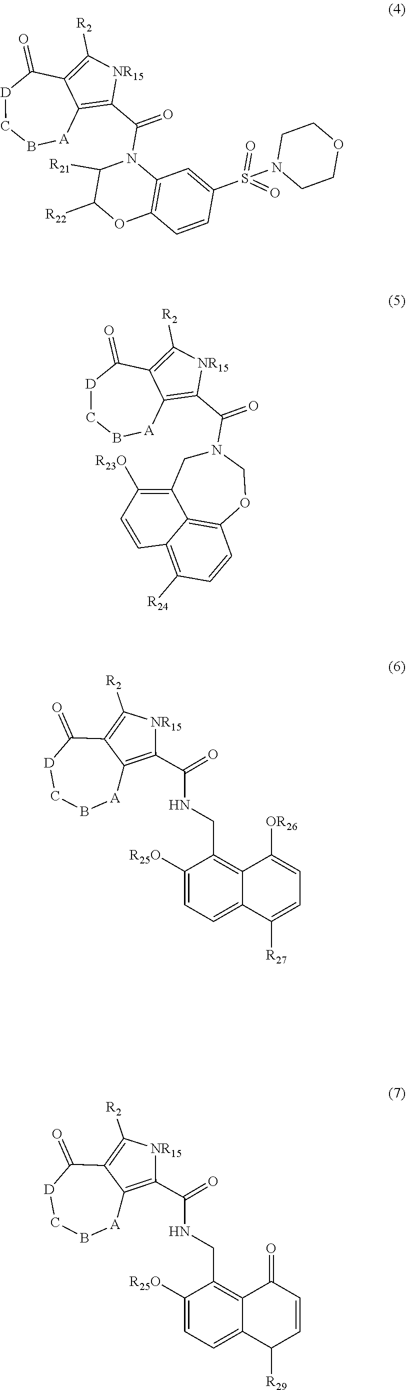
wherein A, B, C, D, R2 and R1s are as defined in claim 1,
R21 and R22, independent of each other, are hydrogen, C1-C4 alkyl or form together a chemical bond,
R23, R2s and R2s, independent of each other, are hydrogen or C1 to C4 alkyl, R24, R27 and R29, independent of each other, are C1-C4 alkyl or C2-C4-carboxyl, and
R26 is hydrogen or C1 to C4 alkyl.

14. The compound of formula (1) or (2) in accordance with claim 13 for use in the treatment of diseases or conditions for which a bromodomain inhibitor is indicated.

15. Use of a compound of formula (1) or (2) in accordance with claim 13 as bromodomain inhibitor.

16. Use of a compound of formula (1) or (2) as defined in claim 13 or a pharmaceutically acceptable salt thereof in the treatment of diseases or conditions selected from chronic autoimmune or inflammatory conditions, cancer or viral diseases, for the inhibition of the proliferation of leukemia cells, for the treatment of obesity, for the treatment of kidney malfunctions, as a male contraceptive, or for the treatment of diseases caused by parasites such as malaria or leishmaniasis.

17. A pharmaceutical composition comprising at least one compound of formula (1) or (2) as defined in claim 13 or a pharmaceutically acceptable salt thereof and one or more pharmaceutically acceptable carriers, diluents or excipients.

18. A combination pharmaceutical product comprising at least one compound of formula (1) or (2) as defined in claim 13 or a pharmaceutically acceptable salt thereof and one or more pharmaceutically acceptable carriers, diluents or excipients together with one or more other pharmaceutically active ingredients.
